(19) United States
(12) Patent Application Publication
(10) Pub. No.: US 2022/0112560 A1
(43) Pub. Date: Apr. 14, 2022

(54) METHODS FOR PREDICTING A VERY LOW BIRTH WEIGHT

(71) Applicant: Imperial College of Science, Technology and Medicine, London (GB)

(72) Inventors: Phillip Bennett, London (GB); Sung Hye Kim, London (GB); David Alan Macintyre, London (GB); Vasiliki Terzidou, London (GB)

(21) Appl. No.: 17/426,057

(22) PCT Filed: Jan. 28, 2020
(86) PCT No.: PCT/GB2020/050190

§ 371 Date: Jul. 27, 2021

(30) Foreign Application Priority Data
Jan. 28, 2019 (GB) 1901120.4

(51) Int. Cl. C12Q 1/6883; C12Q 1/686

(52) U.S. Cl. CPC C12Q 1/6883; C12Q 2600/178; C12Q 1/686

(57) ABSTRACT

The invention provides miRNA biomarkers for the accurate and early prediction or detection of those babies that will be born at a birth weight that is less than 5th centile for the particular gestational age at birth.

### FIELD OF THE INVENTION

The present invention relates to the field of pre-natal diagnostics.

### DESCRIPTION

Fetal growth restriction (FGR) is defined as a fetus who fails to reach its genetically determined growth potential and occurs in both term and preterm babies. It is associated with a higher risk of perinatal morbidity and mortality (Fitzhardinge & Steven, 1972, Low et al., 1978), as well as long-term impacts such as increased incidence of cardiovascular and cerebrovascular diseases (Barker et al, 1989), and non-insulin-dependent diabetes mellitus (Phipps et al., 1993). FGR is often diagnosed by sonographic detection and assessment of fetal weight (Hadlock et al., 1984, Harding et al., 1984).

Babies with less than 10^{th} centile of a reference distribution of weights for the given gestational age are referred as small-for-gestational-age (SGA). It has been estimated that approximately 27% all live births in low- and middle-income countries are born SGA (Lee et al., 2013) which is double the prevalence of low-birth-weight births globally (Black, 2015). That can be due to maternal complications and pre-existing medical conditions (renal disease, antiphospholipid syndrome, chronic hypertension, pre-eclampsia etc), fetal complications or problems with placental function. FGR is not synonymous with SGA (although the terms are sometimes used interchangeably) and represent distinctive clinical conditions. It is important to note that a proportion of infants clinically classified as SGA will have actually grown normally but be constitutionally small, whereas other infants may not have reached their growth potential but have been more than the 10^{th} percentile and are therefore not classified as SGA. The likelihood of FGR is higher in severe SGA infants (less than 5^{th} centile) and placenta dysfunction/placental insufficiency occurs in up to 65% of stillbirths (Phipps et al., 1993). Moreover, the association of SGA with perinatal mortality and major malformations have been reported to be stronger in severe cases of SGA (less than 5^{th} centile) compared to less than 10^{th} centile (Dobson et al., 1981). Current ultrasound-based screening approaches have limited sensitivity and specificity resulting in less than a quarter of SGA babies being identified before birth (Wright et al., 2006). Therefore, there is an urgent need to develop a more refined, reliable early pregnancy risk prediction test.

Early diagnosis or prediction of which babies will be born SGA, particularly those that will be born at a birth-weight that is less than 5^{th} centile, will allow for earlier clinical interventions designed to improve the intrauterine environment and therefore attenuate causes of impaired growth. These include focused monitoring of fetal growth, avoidance of drugs/smoking and control of maternal disorder such as hypertension, adjustments to maternal lifestyle and diet to improve the intrauterine environment, and management of the timing as well as the mode of delivery.

MicroRNAs (miRNAs) are small non-coding RNAs of 19-22 nucleotide in length which regulate the expression of target mRNAs at both the post-transcriptional and translational level (Bartel, 2004). They have been implicated in the regulation of various biological processes including inflammation (Sonkoly & Pivaresi, 2009), cell proliferation and differentiation (Hwang & Mendell, 2006), and apoptosis (Jovanovic & Hengartner, 2006). Growing evidence supports their role in the development and/or management of a wide-range of diseases, which has led to increased interest in the application of miRNAs as potential biomarkers for multitude of human disorders. Pregnancy-related complications are not an exception.

The diagnostic potential of miRNAs have been studied in preeclampsia (Wu et al., 2012), ectopic pregnancy (Zhao et al., 2012), gestational diabetes (Zhao et al., 2011), recurrent pregnancy loss (Hu et al., 2011), preterm delivery (Gray et al, 2017, Winger et al, 2017) as well as SGA. There have been two main approaches to identifying miRNA biomarkers for SGA to date; by looking into differential expression of miRNAs in placenta (Maccani et al., 2011), and that of circulating miRNAs (Rodosthenous et al., 2017).

Due to the relatively non-invasive method of collection and availability, plasma or serum are preferred specimen types for diagnosis of pregnancy-related complications. The majority of the previous work on the identification of miRNA biomarkers for SGA have been focused on the use of technologies such as miRNA microarray and real-time quantitative polymerase reactions (RT-qPCR). These methods have been found to have limited sensitivity in comparison to the digital quantification technology of Nanostring nCounter miRNA profiling assay (Tam et al., 2014). The nCounter assay utilises hybridisation-based method to directly quantify target sequences without the need of an amplification step and is able to quantify less abundant targets such as plasma miRNAs. This study employed the Nanostring nCounter miRNA profiling assay to investigate the potential of circulating plasma miRNAs as a biomarker for small-for-gestational-age births. Despite the lack of a significant correlation between maternal plasma miRNA markers and SGA in the prior art methods, the present inventors have identified robust miRNA markers that can be used to accurately predict that a baby will be born SGA.

### DETAILED DESCRIPTION OF THE INVENTION

The invention provides a method for predicting if a baby will be born at a birth-weight that is less than the 5^{th} centile for the particular gestational age at birth (SGA), wherein the method comprises determining the absolute level, amount or concentration or the relative level, amount or concentration of any one or more of the following miRNAs in a test sample obtained from the mother:
hsa-miR-374a-5p, hsa-let-7d-5p, hsa-miR-4454, hsa-miR-7975, hsa-miR-191, hsa-miR-107, or hsa-miR-30e-5p.

As discussed previously, there is a need for the early and accurate detection of those babies that will be born classed as small for gestational age (SGA). By SGA we include the meaning of babies that are born at less than the 10^{th} centile of the weight of babies born at that particular gestational age. We also include the meaning of babies born at less than the 5^{th} centile of the weight of babies born at that particular gestational age, or less than the 3^{rd} percentile of the weight of babies born at that particular gestational age.

The skilled person will understand what is meant by gestational age. Typically, the gestational age of the fetus is determined by first trimester dating ultrasound scan. This is typically performed in pregnancies at around 12 weeks gestation, as determined by the first day of the mothers last menstrual period. However, other methods are also used, for example based on the date of fertilisation, if known.

By predicting we include the meaning of diagnosing. For example, in one embodiment the invention provides a method for diagnosing a fetus as a fetus that will be born at a birth-weight that is less than the 5^{th} centile for the particular gestational age at birth (SGA), wherein the method comprises determining the absolute level, amount or concentration or the relative level, amount or concentration of any one or more of the following miRNAs in a test sample obtained from the mother:
hsa-miR-374a-5p, hsa-let-7d-5p, hsa-miR-4454, hsa-miR-7975, hsa-miR-191, hsa-miR-107, or hsa-miR-30e-5p.

Each of the miRNAs (hsa-miR-374a-5p, hsa-let-7d-5p, hsa-miR-4454, hsa-miR-7975, hsa-miR-191, hsa-miR-107, or hsa-miR-30e-5p) alone are considered to be useful in predicting whether a baby will be classified as SGA. Accordingly, in one embodiment, the methods of the invention involve determining the amount or level or concentration of any one or more of the following miRNAs in a sample taken from the mother: hsa-miR-374a-5p, hsa-let-7d-5p, hsa-miR-4454, hsa-miR-7975, hsa-miR-191, hsa-miR-107, or hsa-miR-30e-5p.

The skilled person will understand what is meant by reference to the particular miRNAs described herein. For example, the miRNAs discussed herein are well-known and information, including sequence information, can be found in miRNA databases such as http://www.mirbase.org/.

Determining the amount or level or concentration of either hsa-miR-374a-5p or hsa-let-7d-5p (or combination thereof) is considered to have useful predictive power. Accordingly, in one embodiment the methods of the invention involve determining the amount or level or concentration of hsa-miR-374a-5p. In another embodiment the methods of the invention involve determining the amount or level or concentration of hsa-let-7d-5p.

However, it is considered that the method will provide a more accurate prediction when the level, amount or concentration of more than one of the miRNAs is determined. Accordingly, in a preferred embodiment the level, amount or concentration of more than one of the miRNAs is determined in the sample. For example, the level, amount or concentration of at least 1, 2, 3, 4, 5, 6, or all 7 of the miRNAs is determined.

Preferably, in one embodiment the method comprises determining the level, amount or concentration of the following miRNAs:

a) hsa-miR-374a-5p;

b) hsa-let-7d-5p;

c) hsa-miR-374a-5p and hsa-let-7d-5p;

d) hsa-miR-374a-5p and hsa-miR-4454;

e) hsa-miR-374a-5p, hsa-let-7d-5p, hsa-miR-4454, and hsa-miR-7975;

f) has-miR-374a-5p, hsa-miR-4454, hsa-miR-7975, hsa-miR-107 and hsa-miR-30e-5p or

g) hsa-miR-374a-5p, hsa-let-7d-5p, hsa-miR-4454, hsa-miR-7975, hsa-miR-191, hsa-miR-107, and hsa-miR-30e-5p.

The skilled person will understand that a level, amount or concentration of something can be an absolute level, amount, or concentration; or it can be a relative level, amount or concentration. The skilled person will understand whether determination of an absolute level, amount or concentration or a relative level, amount or concentration is required, depending upon the circumstances. Both determining the absolute level, amount or concentration and determining the relative level, amount or concentration are encompassed by the present invention.

By “determining the level, amount or concentration” of the relevant miRNA(s) we include the meaning of determining the absolute amount. For example, in one embodiment the actual physical amount of each miRNA is determined, for example the mass, for example in ng, or the concentration for example ng/ml. Such a method is considered to be particularly useful when the level, amount or concentration needs to be compared to a control sample or control value as discussed below. For example, when performing the method of the invention, once the level, amount or concentration of the particular miRNA(s) has been determined from the maternal sample, it can be directly compared to a level, amount or concentration of that miRNA that is known to be, for example, indicative of the birth of an SGA baby and/or an appropriately grown baby (AGA), i.e. a control level, amount or concentration.

Accordingly, in one embodiment the level, amount or concentration of the miRNA(s) is determined in the sample obtained from the mother, and compared to the level, amount or concentration of the same miRNA(s) in a control sample, or is compared to a control value.

The skilled person will understand that the control sample or control value may be obtained from a sample taken from a mother or a number of mothers who later went on to have babies that either were (positive control) or were not (negative control) classified as SGA. The control sample or value is selected to allow an appropriate comparison to the sample of interest. For example, in one embodiment, the control sample or value is a sample or value from a number of samples taken from mothers that did not have babies classified as SGA, i.e. the babies were AGA. In this instance, a level, amount or concentration of the miRNA(s) of interest that is not similar to the control sample or value and is above or below a certain threshold, relative to the control sample or value would indicate a high likelihood that the baby will be born SGA. Alternatively, the control sample or value is a sample or value from a number of samples taken from mothers that did have babies classified as SGA. In this instance, a level, amount or concentration of the miRNA(s) of interest that is similar to the control sample or value would indicate a high likelihood that the baby will be born SGA. Preferably, the level, amount of concentration of the relevant miRNA(s) from the test sample is compared to both positive and negative controls to allow an accurate prediction to be made.

The selection of appropriate control samples and parameters is well within the capability of the skilled person.

Accordingly, in one embodiment, the control sample is a positive sample that was taken from a pregnant control subject, or a population of pregnant control subjects, who later gave birth to a baby that is SGA, optionally wherein the control sample was taken from the control subject at substantially the same gestation or stage in pregnancy as the gestation or stage at which the test sample is taken from the mother.

In another embodiment, the control sample is a negative sample that was taken from a pregnant control subject, or a population of pregnant control subjects, who later gave birth to a baby that is an appropriately grown baby (AGA), optionally wherein the control sample was taken from the control subject at the same or at substantially the same gestation or stage in pregnancy as the gestation or stage at which the test sample is taken from the mother.

In a preferred embodiment both positive and negative control samples, i.e. control samples taken from a pregnant control subject, or population of pregnant control subjects, who later gave birth to a baby that is SGA and control samples taken from a pregnant control subject, or population of pregnant control subjects, who later gave birth to a baby that is an appropriately grown baby (AGA), optionally wherein the control sample was taken from the control subject at substantially the same gestation or stage in pregnancy as the gestation or stage at which the test sample is taken from the mother, are employed to predict whether the baby will be SGA.

It is preferred if a number of positive and/or negative control samples are used, and the average of these values use to compare against the test value, optionally wherein each control sample was taken from the control subject at the same gestation or stage in pregnancy as the gestation or stage at which the test sample is taken from the mother.

In one embodiment the baby is predicted to be born at a birth-weight that is less than the 5^{th} centile for the particular gestational age at birth if any one or more of the following criteria are met:

a) the amount of hsa-miR-374a-5p is increased relative to the amount of hsa-miR-374a-5p in a control sample, or is increased relative to a control value;

b) the amount of hsa-let-7d-5p is increased relative to the amount of hsa-let-7d-5p in a control sample, or is increased relative to a control value:

c) the amount of hsa-miR-191-5p is increased relative to the amount of hsa-miR-191-5p in a control sample, or is increased relative to a control value;

d) the amount of hsa-miR-4454 is decreased relative to the amount of hsa-miR-4454 in a control sample, or is decreased relative to a control value;

e) the amount of hsa-miR-7975 is decreased relative to the amount of hsa-miR-7975 in a control sample, or is decreased relative to a control value;

f) the amount of hsa-miR-107 is decreased relative to the amount of hsa-miR-107 in a control sample, or is decreased relative to a control value:

g) the amount of hsa-miR-30e-5p is decreased relative to the amount of hsa-miR-30e-5p in a control sample, or is decreased relative to a control value.

In another embodiment the baby is predicted to be born at a birth-weight that is less than the 5^{th} centile for the particular gestational age at birth when all of the following criteria are met:

a) the amount of hsa-miR-374a-5p is increased relative to the amount of hsa-miR-374a-5p in a control sample, or is increased relative to a control value;

b) the amount of hsa-let-7d-5p is increased relative to the amount of hsa-let-7d-5p in a control sample, or is increased relative to a control value;

c) the amount of hsa-miR-4454 is decreased relative to the amount of hsa-miR-4454 in a control sample, or is decreased relative to a control value; and

d) the amount of hsa-miR-7975 is decreased relative to the amount of hsa-miR-7975 in a control sample, or is decreased relative to a control value.

In another embodiment the baby is predicted to be born at a birth-weight that is less than the 5^{th} centile for the particular gestational age at birth when all of the following criteria are met:

a) the amount of hsa-miR-374a-5p is increased relative to the amount of hsa-miR-374a-5p in a control sample, or is increased relative to a control value;

b) the amount of hsa-let-7d-5p is increased relative to the amount of hsa-let-7d-5p in a control sample, or is increased relative to a control value:

c) the amount of hsa-miR-191-5p is increased relative to the amount of hsa-miR-191-5p in a control sample, or is increased relative to a control value;

d) the amount of hsa-miR-4454 is decreased relative to the amount of hsa-miR-4454 in a control sample, or is decreased relative to a control value:

e) the amount of hsa-miR-7975 is decreased relative to the amount of hsa-miR-7975 in a control sample, or is decreased relative to a control value; f) the amount of hsa-miR-107 is decreased relative to the amount of hsa-miR-107 in a control sample, or is decreased relative to a control value; and

g) the amount of hsa-miR-30e-5p is decreased relative to the amount of hsa-miR-30e-5p in a control sample, or is decreased relative to a control value.

As discussed above, the absolute level, amount or concentration of the miRNA(s) of the test sample can be compared to a standard control level, amount or concentration of the same miRNA(s). However, it will also be apparent that the relative level, amount or concentration of the same or different miRNA(s) can be used to predict the likelihood that a baby will be born SGA.

The skilled person will appreciate that whilst it is of course possible to determine the actual absolute level, amount or concentration of each of the relevant miRNAs in samples taken at both the earlier and later times points, and that these absolute levels may be compared either to each other to determine an increase or decrease, or compared to a control sample or value as discussed above, since the samples are taken from the same individual, it may not be necessary to use any external control samples or values to arrive at a prediction of SGA. This is particularly true where the two samples are processed at the same time. In this instance it may not even be necessary to normalize the data to internal “housekeeping” normalization miRNAs or genes.

The skilled person will also realise that using a ratio of two more of the miRNAs from a single sample to predict SGA has significant advantages, including simplicity and a lack of necessary external control samples or values and concomitant potential error in comparison to external controls. The inventors have surprisingly found that the ratio of the amounts of two of the miRNAs has particularly strong predictive power. Accordingly, in one embodiment, the ratio of any 1, 2, 3, 4, 5, 6, or 7 of the relative expression or absolute amounts of any of the hsa-miR-374a-5p, hsa-let-7d-5p, hsa-miR-191-5p, hsa-miR-4454, hsa-miR-7975, hsa-miR-107, and hsa-miR-30e-5p miRNAs is calculated and used to determine or predict if a baby will be born at a birth-weight that is less than the 5^{th} centile for the particular gestational age (SGA),

In a preferred embodiment, the relative expression or absolute amounts of hsa-miR-374a-5p and hsa-miR-4454 are determined and the ratio of hsa-miR-374a-5p to hsa-miR-4454 is calculated and used to determine or predict if a baby will be born at a birth-weight that is less than the 5^{th} centile for the particular gestational age at birth (SGA),

The skilled person will understand that the average expression level or amount of any 1, 2, 3, 4, 5, or 6 of the miRNAs can be used in a ratio with the expression level or amount or any 1, 2, 3, 4, 5 or 6 of the other miRNAs. In yet a further embodiment, the average expression level or amount of any 1, 2, 3, 4, 5, or 6 of the miRNAs can be used in a ratio with the average expression level or amount or any 1, 2, 3, 4, 5 or 6 of the other miRNAs.

Accordingly, in another embodiment, the ratio of the relative expression or absolute amounts of hsa-miR-374a to the average relative expression or absolute amounts of all 4 miRNAs that decrease in SGA cases (hsa-miR-30e, hsa-miR-107, hsa-miR-4454 and hsa-miR-7975) is calculated and used to determine or predict predicting if a baby will be born at a birth-weight that is less than the 5^{th} centile for the particular gestational age at birth (SGA), In one embodiment, the method further comprises determining the ratio of the relative expression of hsa-miR-374a-5p to hsa-miR-4454.

In preferred embodiments, the relative amounts of the miRNAs are used to determine the ratios rather than the absolute amounts, since in some embodiments this is considered to negate the requirement for an external control sample or value.

In this embodiment it is not considered necessary to determine the absolute level, amount or concentration of these miRNAs, particularly when the amount of each miRNA is determined at the same time so the influence of variations in methods or reagents etc would be expected to affect the determined amount of each miRNA similarly.

As above, it is of course possible to determine the absolute level, amount or concentration of the miRNAs, for example the hsa-miR-374a-5p to hsa-miR-4454 miRNAs, and calculate the ratio.

It is also possible to determine the absolute level, amount or concentration of the miRNAs, for example the hsa-miR-374a-5p to hsa-miR-4454 and calculate the ratio in the test sample and compare this to a ratio derived from a control sample or samples.

In one embodiment, the baby is predicted to be born at a birth-weight that is less than the 5^{th} centile for the particular gestational age at birth if the ratio of the amount of hsa-miR-374a-5p to hsa-miR-4454 is 1.5 or more, optionally at least 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9. 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0.

The method of the invention can be performed on a sample obtained from any animal, preferably on any mammal, preferably on a human. In a preferred embodiment, the method is performed on a sample obtained from a human.

The sample from the mother (and indeed the control sample where appropriate) can be any type of sample. In one preferred embodiment, the test sample is a blood sample, or is a fraction of a blood sample, for example is a serum sample, plasma sample and/or blood-derived exosomes. A blood or plasma sample can be obtained by routine methods known in the art.

The sample can be taken from the mother at any stage during pregnancy. For example, in one embodiment the sample is taken during the first trimester. In another embodiment the sample is taken during the second trimester. In a further embodiment, the sample is taken during the third trimester.

In another embodiment, the test sample is taken from the mother at between around 12⁺⁰ weeks gestation and 21⁺⁶ weeks gestation, optionally between 12⁺⁰ and 14⁺⁶, or 15⁺⁰ and 17⁺⁶, or 18⁺⁰ and 21⁺⁶ weeks gestation.

In one advantageous embodiment the test sample is taken between 12⁺⁰ and 14⁺⁶ weeks of gestation. This coincides with the timing of other routine tests, and prediction or detection of those babies that are expected to be born at a birth-weight that is less than 5^{th} centile at this early stage in pregnancy is considered to offer the best outcomes for the baby.

The absolute or relative level, amount, concentration of the miRNA(s) can be determined using any method available to the skilled person. Such methods are routine and the selection of an appropriate method will be well within the capability of the skilled person.

In one embodiment, the level of the miRNA(s) is determined using digital molecular barcoding technology, optionally Nanostring technology, optionally the Nanostring nCounter miRNA profiling assay, as discussed in, for example, Example 4. Other methods of miRNA(s) quantification include microarray, next generation sequencing platforms, in situ hybridization and RT-PCR. Some of these, such as RT-PCR, may further comprise determining the amount of one or more suitable normalisation miRNAs present in the sample. Again, the Examples provide more information on the use of RT-PCR. For example, in one embodiment the normalisation miRNAs are preferably stable across experimental conditions and/or disease states. In one embodiment the normalisation miRNAs are hsa-miR-30d-5p and/or hsa-let-7i-5p. Routinely used housekeeper genes such as GAPDH and β-actin may also be used as a means to quantify relative expression of miRNA(s). The housekeeper genes are constitutively expressed genes that are often required for the maintenance and basic cellular function, the expression of which does not vary across experimental conditions and/or disease state.

In another embodiment, the method comprises determining the amount of any one or more of hsa-miR-374a-5p, hsa-let-7d-5p, hsa-miR-4454, and hsa-miR-7975 miRNAs using RT-PCR, optionally further comprising determining the level of hsa-miR-30d-5p and/or hsa-let-7i-5p miRNAs using RT-PCR.

In another embodiment the method comprises determining the amount of hsa-miR-374a-5p and hsa-miR-4454 using RT-PCR, optionally further comprising determining the level of hsa-miR-30d-5p and/or hsa-let-7i-5p miRNAs using RT-PCR, optionally further comprising determining the ratio of hsa-miR-374a-5p to hsa-miR-4454.

In another embodiment the method comprises determining the amount of hsa-miR-374a-5p, hsa-let-7d-5p, hsa-miR-4454, and hsa-miR-7975 using RT-PCR, optionally further comprising determining the level of hsa-miR-30d-5p and/or hsa-let-7i-5p miRNAs using RT-PCR.

In another embodiment the method comprises determining the amount of hsa-miR-374a-5p, hsa-let-7d-5p, hsa-miR-4454, hsa-miR-7975, hsa-miR-191, hsa-miR-107, and hsa-miR-30e-5p using RT-PCR, optionally further comprising determining the level of hsa-miR-30d-5p and/or hsa-let-7i-5p miRNAs using RT-PCR.

It will be apparent to the skilled person that once an early prediction of SGA has been made using the methods of the invention, appropriate interventions can be made to mitigate negative consequences of an SGA classification. Accordingly, in one embodiment the method further comprises treating the mother and/or baby for SGA.

Such therapeutic treatments include, as discussed previously, prenatal interventions to improve intrauterine environment/attenuating causes of impaired growth, which include improved nutrition, avoidance of drugs/smoking and control of maternal disorder such as hypertension. Other treatments include administration of aspirin, increased surveillance of the fetus, and determining optimal timing and the mode of delivery of the baby.

Accordingly in one embodiment the method further comprises treating the mother with aspirin, nutritional supplements, anti-hypertension therapeutics and arranging earlier delivery of the baby, i.e. ahead of the delivery that would otherwise have occurred naturally at a later time point.

Accordingly, the invention provides a method for treating a mother, or for treating a baby, for determined or predicted fetal birth-weight that is at less than 5^{th} centile for the particular gestational age at birth, wherein the method comprises determining or predicting that the baby will be born at a birth-weight that is less than 5^{th} centile for the particular gestational age at birth. The method may further comprise treating the mother and/or baby with aspirin, nutritional supplements, anti-hypertension therapeutics and earlier delivery of the baby, i.e. ahead of the delivery that would otherwise have occurred naturally at a later time point.

The invention also provides aspirin, nutritional supplements and anti-hypertension therapeutics for use in treating a baby and/or mother for determined or predicted birth at a birth-weight that is at less than 5^{th} centile for the particular gestational age at birth, wherein the baby has been determined or predicted to be born at a birth-weight that is less than 5^{th} centile for the particular gestational age at birth using the method of the invention.

The invention also provides the use of nutritional supplements, anti-hypertension therapeutics, and aspirin for use in the manufacture of a medicament for treating a baby and/or mother for determined or predicted birth at a birth-weight that is less than 5^{th} centile for the particular gestational age at birth, wherein the baby has been determined or predicted to be born at a birth-weight that is less than 5^{th} centile for the particular gestational age at birth using the method of the invention.

The invention also provides a method of treating a baby that was born at a birth-weight that is less than the 5^{th} centile for the particular gestational age at birth to increase growth wherein the baby was predicted to be born at a birth-weight that is less than the 5^{th} centile for the particular gestational age at birth using the method of predicting if the baby will be born at a birth-weight that is less than the 5^{th} centile for the particular gestational age at birth of the invention.

The invention also provides a kit comprising the means to detect the absolute or relative level, amount or concentration of any one or more of the following miRNAs:

hsa-miR-374a-5p, hsa-let-7d-5p, hsa-miR-4454, hsa-miR-7975, hsa-miR-191, hsa-miR-107, and/or hsa-miR-30e-5p.

In another embodiment, the kit comprises means to detect the amount of hsa-miR-374a-5p and hsa-miR-4454.

In another embodiment, the kit comprises means to detect the amount of hsa-miR-374a-5p, hsa-let-7d-5p, hsa-miR-4454 and hsa-miR-7975.

In another embodiment, the kit comprises means to detect the amount of all of hsa-miR-374a-5p, hsa-let-7d-5p, hsa-miR-4454, hsa-miR-7975, hsa-miR-191, hsa-miR-107, and hsa-miR-30e-5p.

It will be clear to the skilled person that the detection of particular miRNAs can be performed using an oligonucleotide with a sequence that is complementary to at least part of the miRNA nucleic acid sequence, for example by using an oligonucleotide primer or probe. Accordingly in one embodiment the means to detect the amount of the miRNAs are oligonucleotides, optionally wherein at least a part of the sequence of the oligonucleotide is complementary to at least part of the miRNA nucleic acid sequence.

Other means to detect the miRNAs include antibody based means, for example antibodies or antibody fragments.

In some embodiments, the means to detect the amount of the miRNAs are immobilised to a solid surface. For example, in one embodiment the kit comprises at least two oligonucleotides, optionally wherein at least a part of the sequence of each oligonucleotide is complementary to at least part of one of the miRNA nucleic acid sequences, wherein the oligonucleotides are immobilised to a solid surface.

In one embodiment the kit is suitable for use at the bedside.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. For example, the invention provides a method for predicting if a baby will be born at a birth-weight that is less than the 5^{th} centile for the particular gestational age at birth, wherein the method comprises determining the level, amount or concentration of hsa-miR-374a-5p, hsa-let-7d-5p, hsa-miR-4454, and hsa-miR-7975, wherein the method further comprises comparing the level, amount or concentration of each of the miRNAs to a control level, amount or concentration of the same miRNAs that is the average level, amount or concentration of the miRNA derived from a number of samples taken from mothers who later gave birth to an SGA baby.

The invention also provides a method for predicting if a baby will be born at a birth-weight that is less than the 5^{th} centile for the particular gestational age at birth, wherein the method comprises determining the relative amounts of hsa-miR-374a-5p and hsa-miR-4454 in a sample taken from the mother and calculating the ratio of hsa-miR-374a-5p to hsa-miR-4454, and predicting that the baby will be born SGA if the ratio is at least 4.4.

### FIGURE LEGENDS

FIG. 1. Expression of the top 50 plasma miRNAs. Hierarchical clustering heat map showing expression profiles for top 50 highly expressed plasma miRNAs from SGA and controls. Heat map was built based on normalised nCounter array probe counts.

FIG. 2. Multivariate modelling of plasma miRNA expression of women who 35 delivered SGA babies and those with normal weight babies. (a) Principal component analysis (PCA) score plot of top 50 expressed plasma miRNAs. (b) Partial least squares discriminant analysis (PLS-DA) score plot shows clear clustering of SGA and control groups. (c) Validation of the PLS-DA model by comparison to the classification statistics of models generated after 100 random permutations of the data showing degradation of R² to below 0.3 and Q² to below 0. (d) Variable importance in the projection (VIP) plot of the PLS-DA model represents a panel of miRNAs according to the importance of miRNA in discriminating between miRNA profiles of the two groups. (e) Regression coefficient plots of miRNA variables in the PLS-DA model indicating most important miRNA expression changes (increase/decrease) in SGA compared with controls.

FIG. 3. Top 7 miRNA profiles in maternal plasma at time point A (12⁺⁰-14⁺⁶ weeks). Graphs show probe counts of endogenous miRNAs, hsa-miR-374a-5p (a), hsa-miR-191 (b), hsa-let-7d-5p (c), hsa-miR107 (d), hsa-miR-30e-5p (e), and hsa-miR-4454+hsa-miR-7975 (f), from human maternal plasma at time point A (12⁺⁰-14⁺⁶ weeks) in women who went on to deliver SGA babies or normal weight babies (Control n=16, SGA n=11, Mann-Whitney U test).

FIG. 4. Technically validated miRNAs differentiating SGA and control samples at time point A (12⁺⁰-14⁺⁶ weeks) in the discovery cohort. Total RNA were extracted from plasma samples and used to validate candidate miRNA expression using RT-qPCR. Resulting Cq values normalised for any discrepancies in the efficacy of RNA extraction using the spike-in cel-254 (5000 attomoles) and for reverse transcription using the spike-in UniSp6. Two miRNAs, hsa-miR-30d-5p and hsa-let-7i-5p, identified using NormFinder were used as endogenous controls. All values were then normalised relative to the average of the control samples. P values were determined by Mann-Whitney U-test. The differential plasma levels of hsa-miR-374a-5p (a), hsa-let-7d-5p (b), hsa-miR-4454 (c) and hsa-miR-7975 (d) were validated using RT-qPCR. Hsa-miR-191 (e), hsa-miR107 (f) and hsa-miR-30e-5p (g) in SGA patients were not significantly different compared to normal controls (Control n=16, SGA n=11, Mann-Whitney U test).

FIG. 5. Expression ratio of technically validated miRNAs to differentiate SGA cases and normal controls. Relative expressions of candidate miRNAs obtained from RT-qPCR were used to obtain the ratio of hsa-miR-374a-5p to hsa-miR-4454. ROC analysis of expression ratios of miR-374a-5p to hsa-miR-4454 exhibited strong predictive ability for SGA cases with AUC of 0.97 (b). Similar data is shown for the ratio of miR-374a-5p to the average of all 4 miRNAs that decrease in SGA cases (miR-30e, miR-107, miR-4454 and miR-7975) (c and d).

FIG. 6. Process flow chart for miRNAs extraction from plasma. Simple workflow is designed to extract and purify plasma miRNAs from whole blood samples of pregnant women at antenatal clinic. Serial plasma samples were processed for nCounter assay to identify potential miRNA biomarkers and for technical validation experiments.

FIG. 7. Multivariate modelling of plasma miRNA expression of women who delivered SGA babies and those with normal weight babies at each time-point. (a) Principal component analysis (PCA) score plots of top 50 expressed plasma miRNAs at time-point A (TPA), B (TPB) and C (TPC). (b) Partial least squares discriminant analysis (PLS-DA) score plots demonstrate clear clustering of SGA and control groups at time-point A, B and C. (c) Loadings plot at each time-point shows miRNAs that contribute to the discrimination of SGA cases and controls. (d) Validation of the PLS-DA models were carried out by data permutations (100 permutations).

FIG. 8. Top 7 miRNA profiles in maternal plasma at time point B and C. Graphs show probe counts of endogenous miRNAs, hsa-miR-374a-5p (a), hsa-miR-191 (b), hsa-let-7d-5p (c), hsa-miR107 (d), hsa-miR-30e-5p (e), and hsa-miR-4454+hsa-miR-7975 (f), from human maternal plasma at time point B and C in women who went on to deliver SGA babies or normal weight babies.

FIG. 9. Expression of previously reported miRNA markers for FGR. Graphs show nCounter probe counts of endogenous miRNAs, hsa-miR-21-5p (a), hsa-miR-194-5p (b), hsa-574-3p (c), hsa-miR-518b (d), hsa-miR-525-5p (e), hsa-miR-141-3p (f), hsa-miR-499a-5p (g) and hsa-miR-424-5p (h) from human maternal plasma at time point B and C in women who went on to deliver SGA babies or normal weight babies.

FIG. 10. Validation of miRNAs differentiating SGA and control samples at 12-14⁺⁰ weeks in an independent cohort. Total RNA were extracted from plasma samples and used to validate candidate miRNA expression using RT-qPCR. Resulting Cq values normalised for any discrepancies in the efficacy of RNA extraction using the spike-in cel-254 (5000 attomoles) and for reverse transcription using the spike-in UniSp6. Two miRNAs, hsa-miR-30d-5p and hsa-let-7i-5p, identified using NormFinder were used as endogenous controls. All values were then normalised relative to the average of the control samples. P values were determined by Mann-Whitney U-test. The differential plasma levels of hsa-miR-374a-5p (a), hsa-let-7d-5p (b), hsa-miR-4454 (c) and hsa-miR-7975 (d) were validated using RT-qPCR (Control n=83, SGA n=12, Mann-Whitney U-test). ROC analysis of relative expression of hsa-miR-374a-5p and hsa-let-7d-5p exhibited good predictive ability for SGA cases with AUC greater than 0.70.

FIG. 11. Combination of relative expression of hsa-miR-374a-5p, hsa-let-7d-5p, hsa-miR-4454 and hsa-miR-7975 improves predictive ability for SGA cases. The combination of relative expressions of hsa-miR-374a-5p, hsa-let-7d-5p, hsa-miR-4454 and hsa-miR-7975 obtained from RT-qPCR were used for ROC curve analysis. Combination of these relative expressions enhanced the predictive ability for SGA cases with AUC of 0.753.

### EXAMPLES

### Example 1

Elucidation of Plasma miRNA Profiles in SGA Cases and Controls

There was a total of 29 participants recruited of whom 13 went on to have SGA babies and 16 normal weight. The demographic and clinical characteristics of the participants are illustrated in Table 1. As expected, there was a significant difference in the birth-weights of SGA cases compared to normal controls (p<0.001) and no differences were observed in the other matching variables.

TABLE 1
Demographic and clinical characteristics of study cohort.
Demographic and Clinical variables

| | | Controls | | SGA | | p- |
|---|---|---|---|---|---|---|
| | | (n = 16) | | (n = 13) | | value* |
| Maternal age (SD) | | 35.9 | (5.88) | 32.9 | (5.91) | 0.1804 |
| Ethnicity (%) | Caucasian | 8 | (50) | 8 | (61.5) | 0.1145 |
| | Arab | 3 | (18.75) | 1 | (7.7) | |
| | Black | 4 | (25) | 3 | (23.1) | |
| | Oriental | 1 | (6.25) | 1 | (7.7) | |
| Gestational age | | 38.6 | (4.24) | 37.7 | (3.61) | 0.1105 |
| at delivery (SD) | | | | | | |
| Baby gender | Male | 7 | (43.75) | 7 | (53.85) | 0.2029 |
| (%) | Female | 9 | (56.25) | 6 | (46.15) | |

| | | | |
|---|---|---|---|
| Birthweight | 3455 | 2190 | <0.0001 |

*p-value corresponds to Mann-Whitney U test (continuous) or Chi-squared (categorical) for the difference in study participants' characteristics between control and SGA groups.

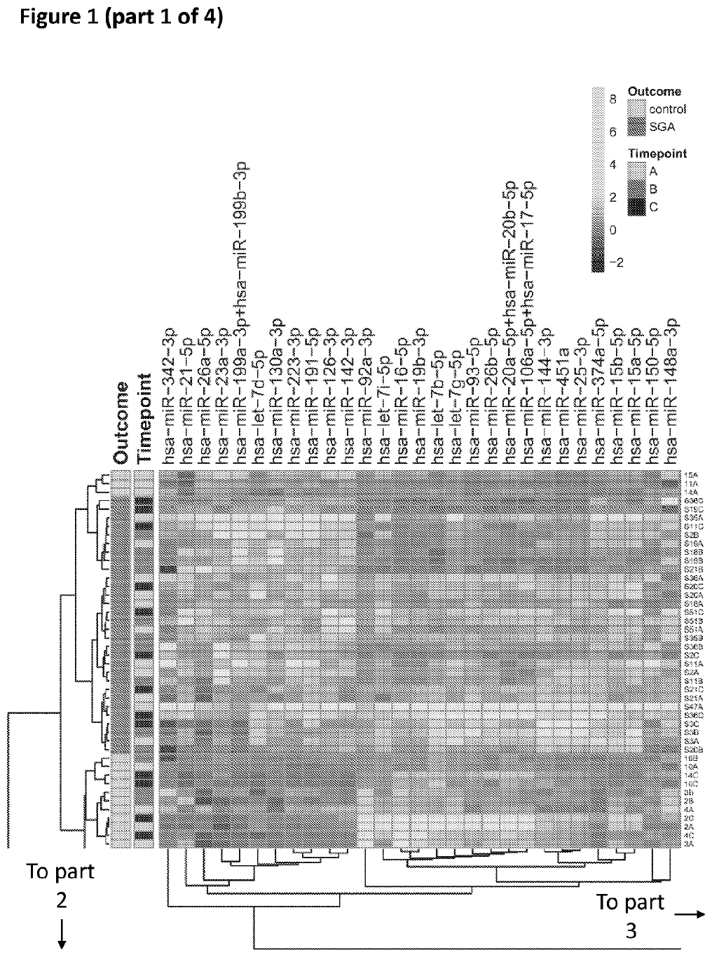

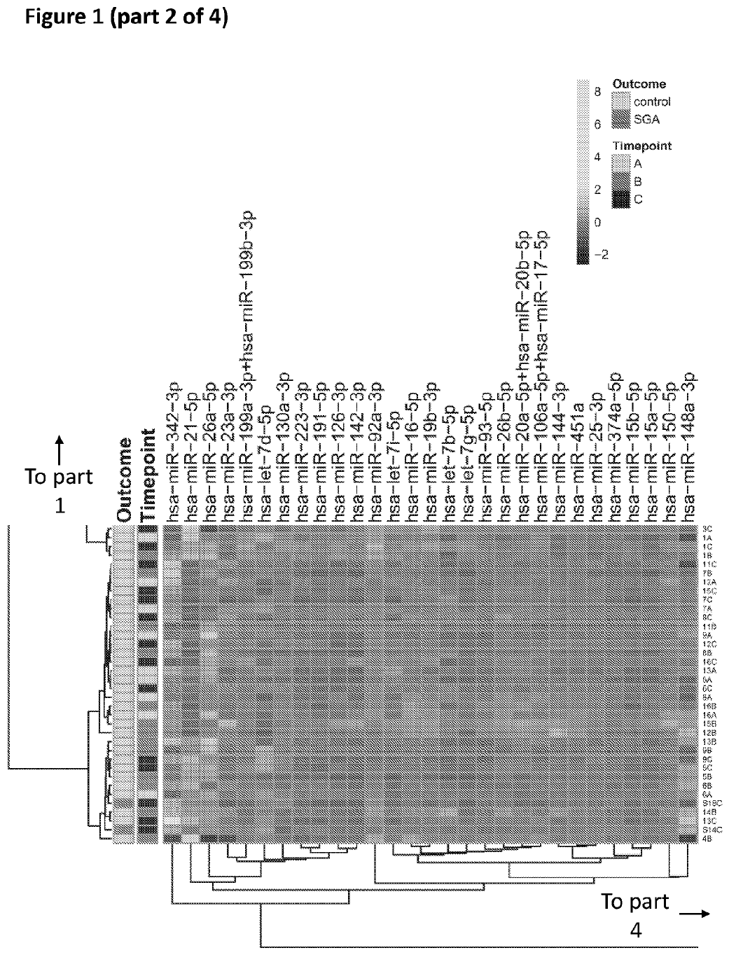

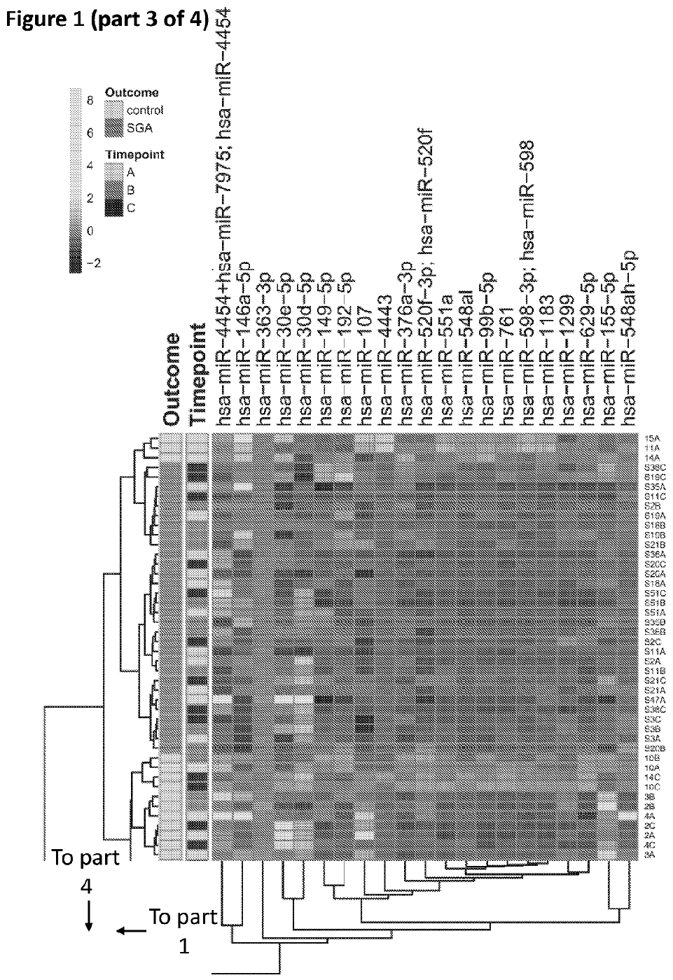

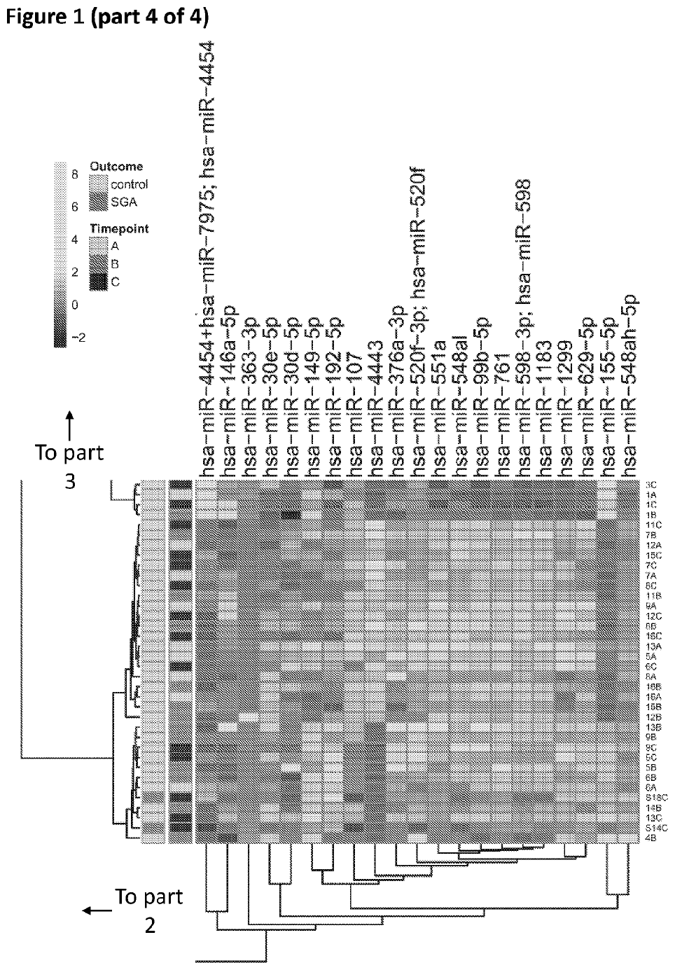

The nCounter miRNA profiling assay examined the expression of 800 plasma miRNAs from which 414 miRNAs were found to be expressed above background. The top 50 highly expressed miRNAs were used for further analysis. Hierarchical clustering of the top 50 highly expressed miRNAs showed a clear separation of SGA cases from controls (FIG. 1). There were groups of miRNAs that were up-regulated in SGA compared to controls and those that were down-regulated.

Identification of Plasma miRNA Markers Characterising SGA Cases

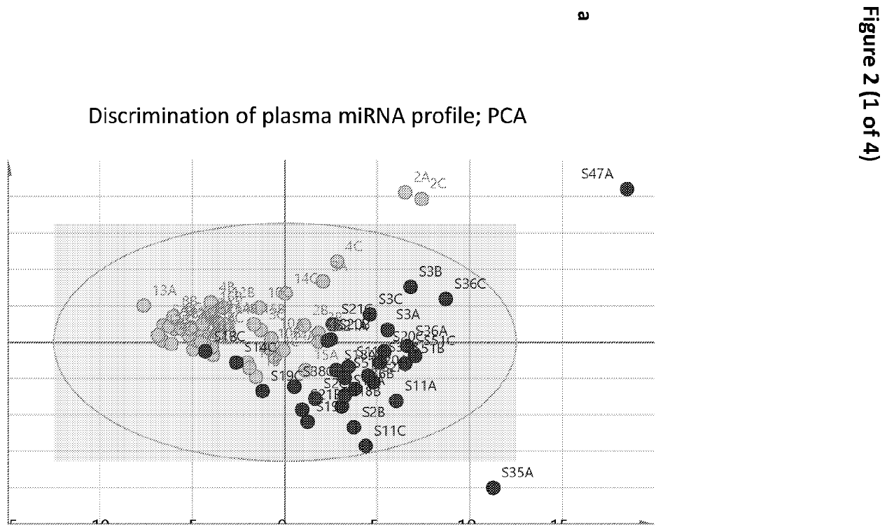

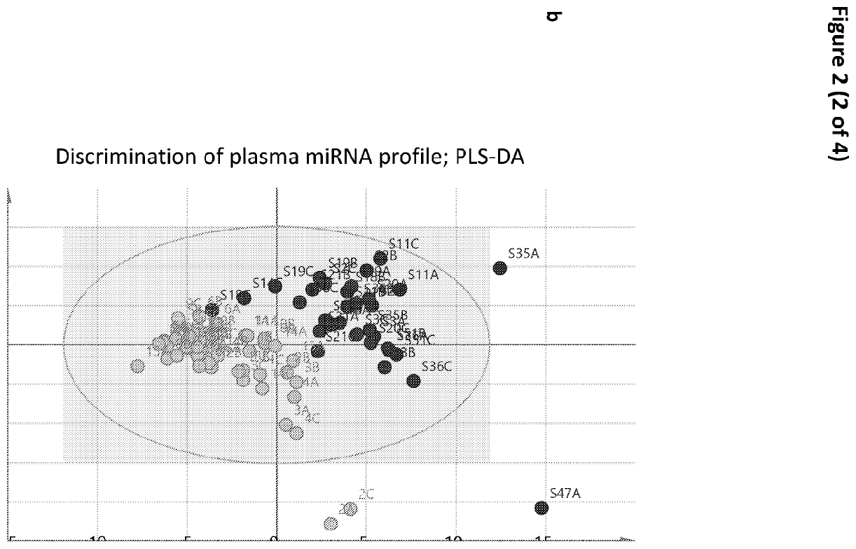

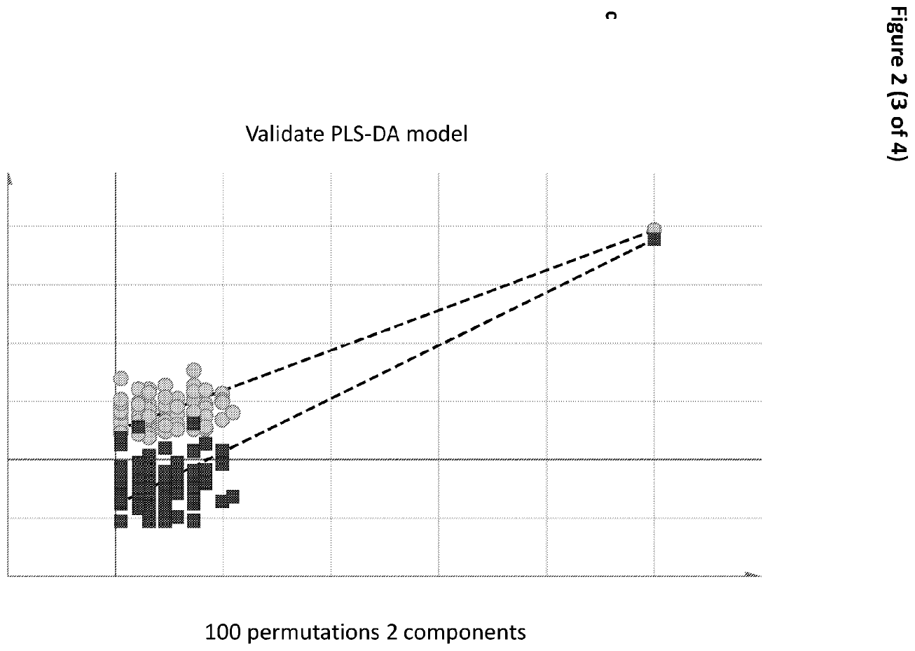

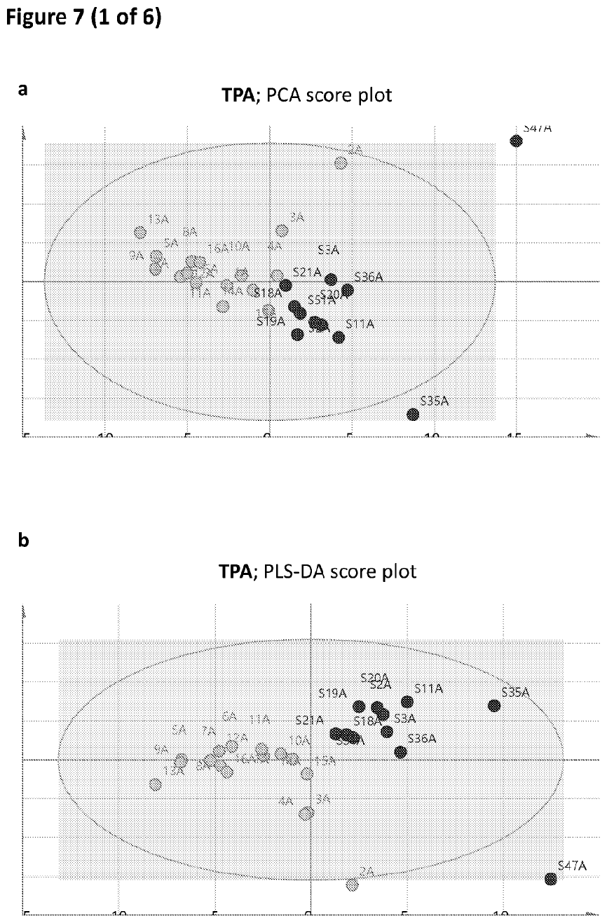

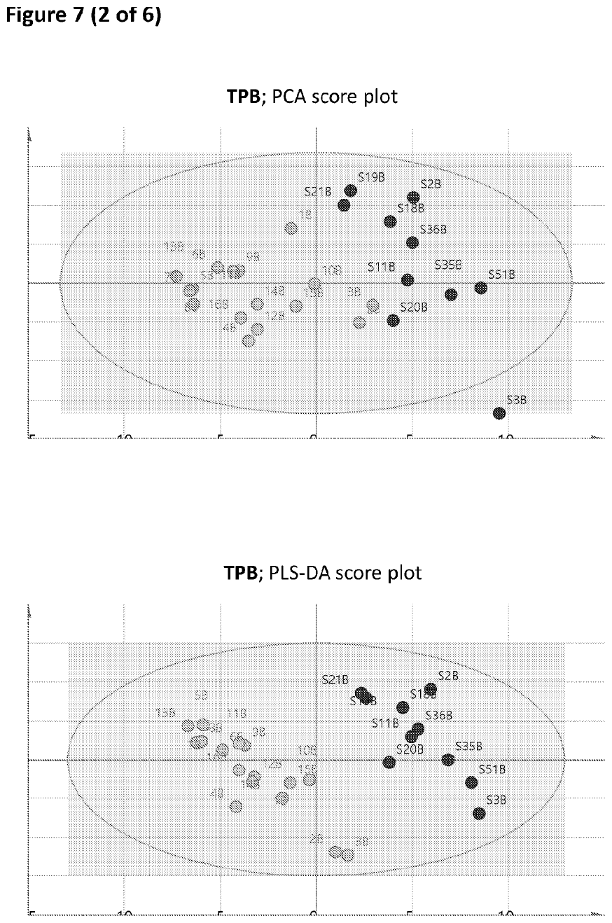

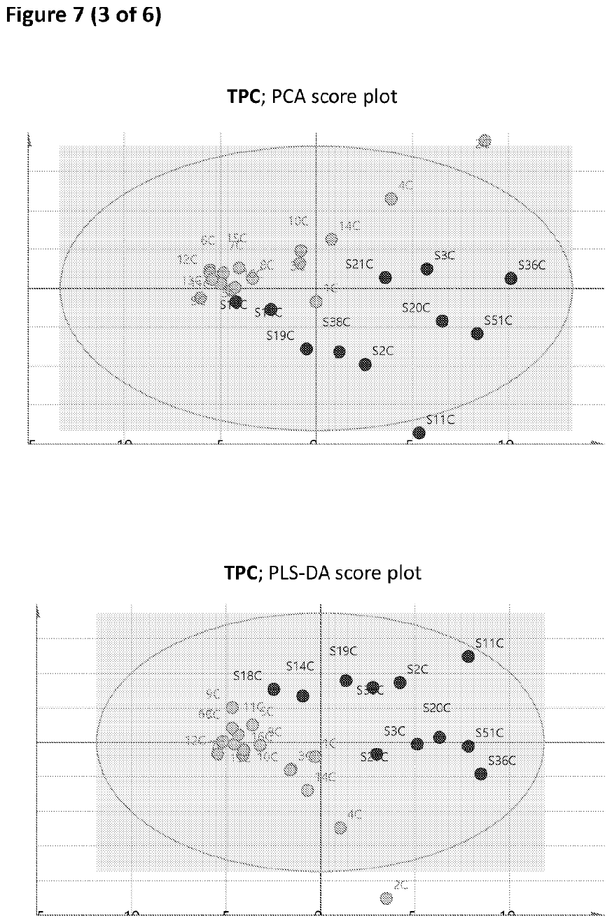

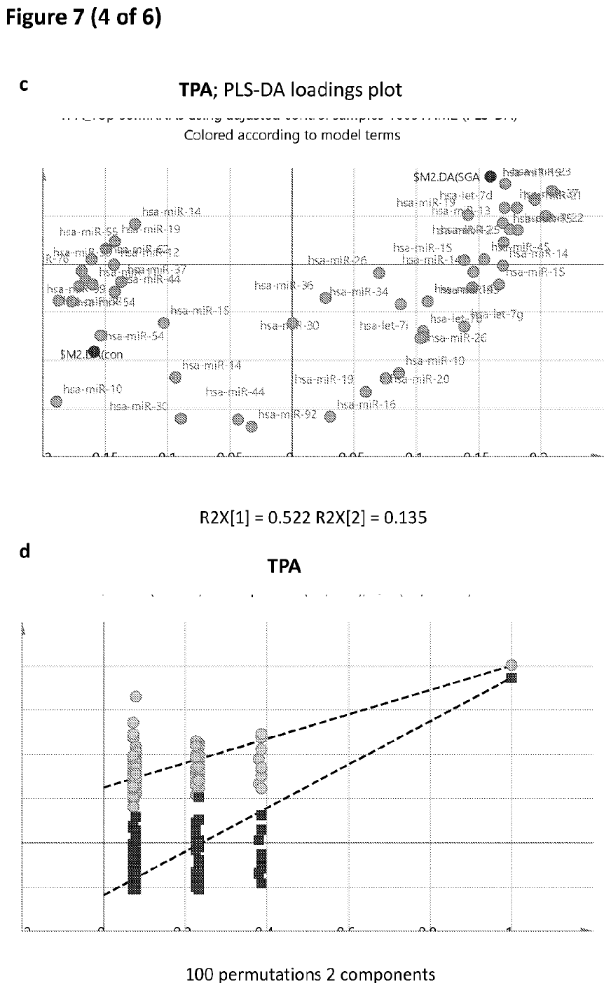

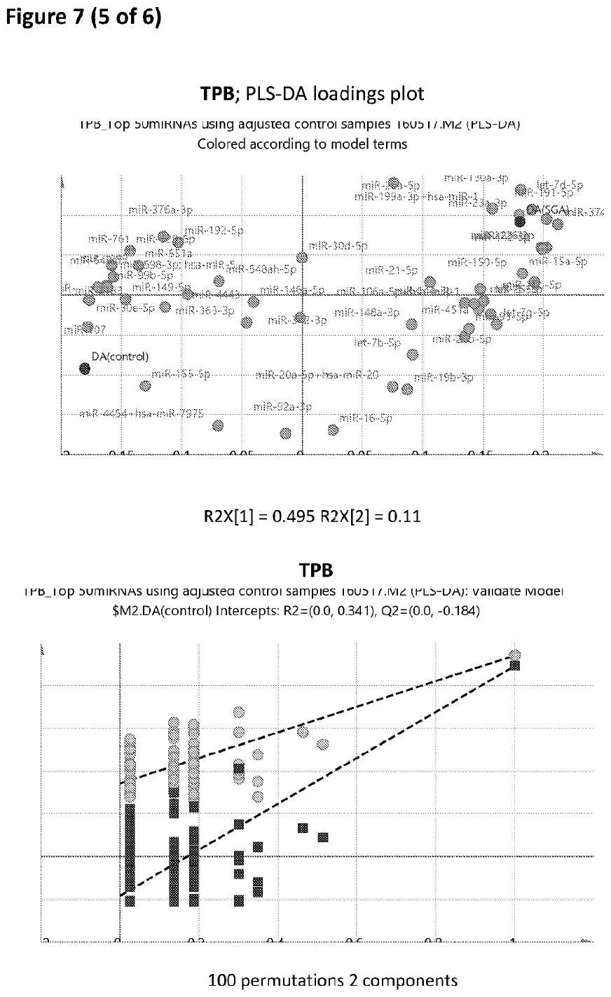

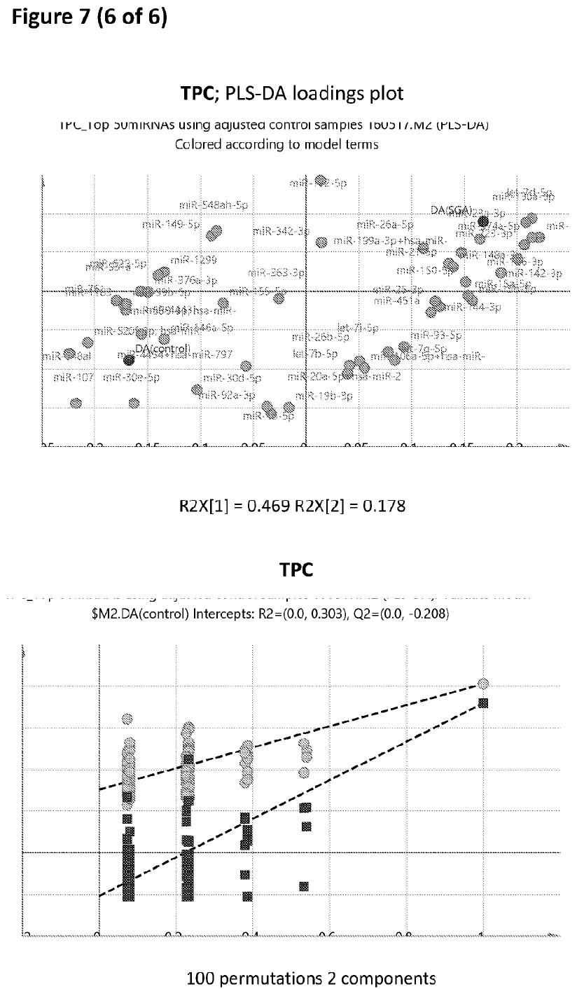

The profile data from the top 50 highly expressed plasma miRNAs were used for multivariate analysis. Unsupervised principal component analysis (PCA) of the first two components was carried out to ascertain the data clustering, outliers and trends. PCA scores plot showed the clustering of SGA cases and controls (FIG. 2a). Following this, supervised partial least-squares discriminant analysis (PLS-DA) was applied for group discrimination. The miRNA profiles of SGA cases were clearly distinct from those of controls and clustering was observed between the two groups as shown in the PLS-DA scores plot (FIG. 2b). The predictive PLS-DA model produced was robust (R²(X)=0.619, R²(Y)=0787, Q²=0.756 for the two components) (Atherton et al, 2009) and this model was cross validated using data permutations. After 100 permutations, the values of R² (correlation) and Q² (predictability) degraded from >0.7 to 0.0997 and −0.155, respectively (FIG. 2c), which demonstrates that the data were not overmodelled in PLS-DA (Semmo et al., 2015). Based on this model, the significant miRNAs in group classification were obtained using combination of Variable Importance in the Projection (VIP) of >1 and regression coefficients plot where larger coefficient values (positive/negative) demonstrate stronger correlation with miRNA profile discrimination (FIGS. 2d and e). Following multivariate analysis at each time-point (FIG. 7), 6 nCounter probes, for 7 miRNA targets, were identified to contribute the most to the discrimination of SGA cases and controls at all time-points (Table 2) which includes the top 3 probes, hsa-miR-374a-5p, hsa-miR-191-5p, and hsa-let-7d-5p, upregulated in SGA cases compared to controls and the top 3 probes for hsa-miR-107, hsa-miR-30e-5p and hsa-miR-4454+miR-7975 that are downregulated in SGA cases (FIG. 2e).

TABLE 2
miRNAs differentially expressed in human plasma in SGA
cases compared to controls at different time points. The miRNAs
listed were identified by the nCounter miRNA profiling assay data.
Target miRNAs significant in classification (outcome)

| 50miRNAs | 50miRNAs | 50miRNAs | 50miRNAs | miRNA |
|---|---|---|---|---|
| (All TPs) | (TPA) | (TPB) | (TPC) | counts |
| miR-374a | miR-374a | miR-374a | miR-374a | |
| Let-7d | miR-23a | Let-7d | Let-7d | High in SGA |
| miR-191 | miR-191 | miR-191 | miR-191 | |
| miR-107 | miR-107 | miR-155 | miR-107 | |
| miR-30e | miR-30e | miR-16 | miR-30e | High in |
| | | | | Controls |
| miR-4454 + | miR-4454 + | miR-4454 + | miR-548al | |
| miR-7975 | miR-7975 | miR-7975 | | |

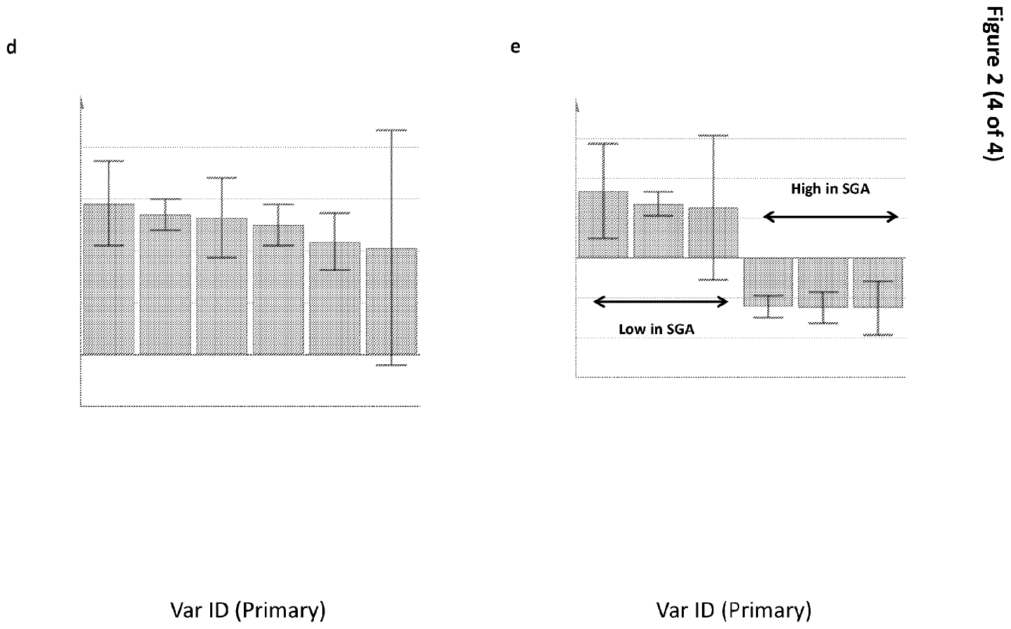

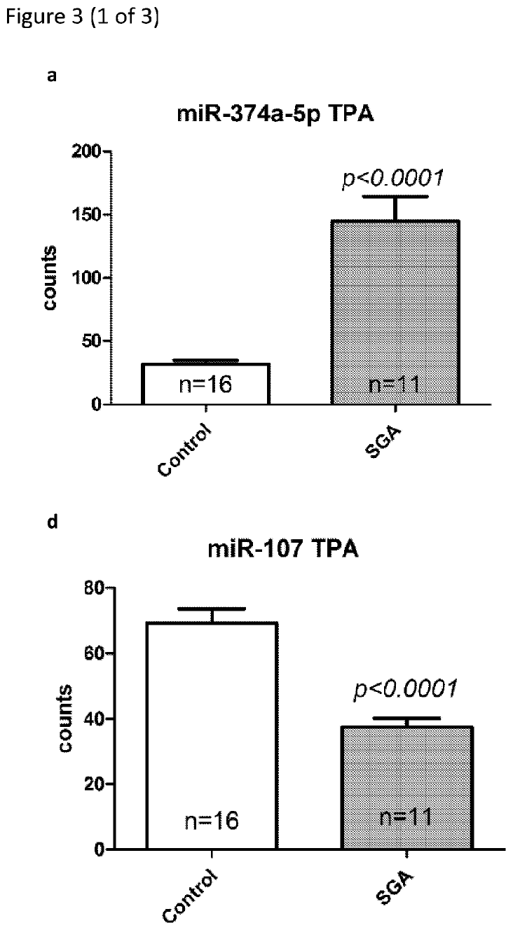

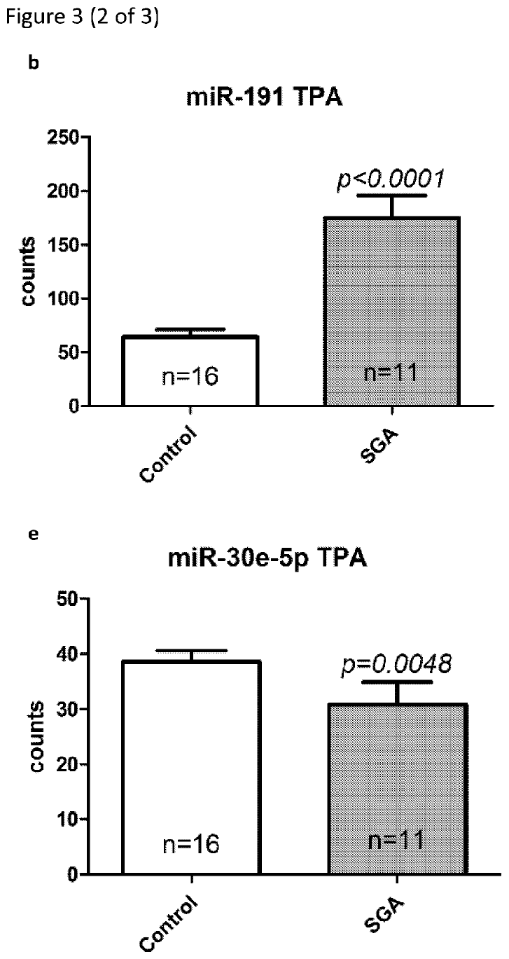

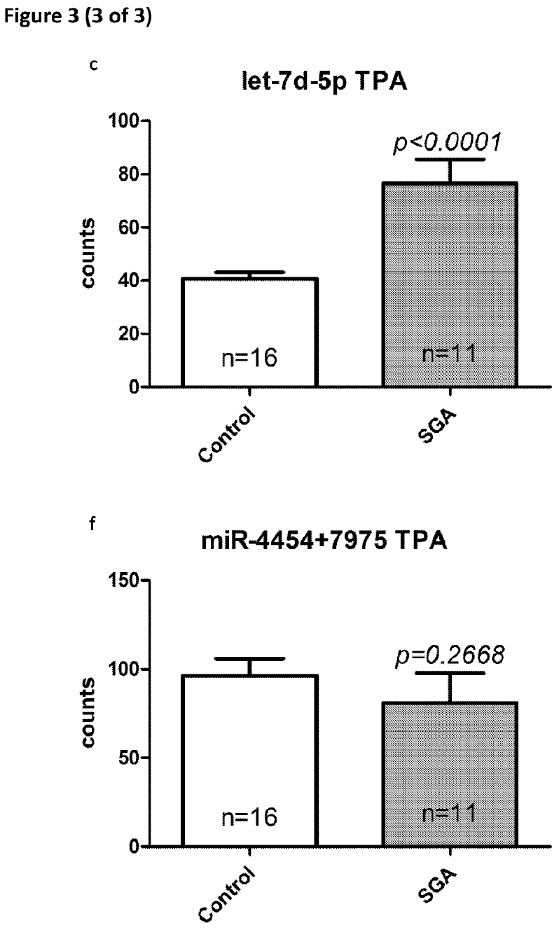

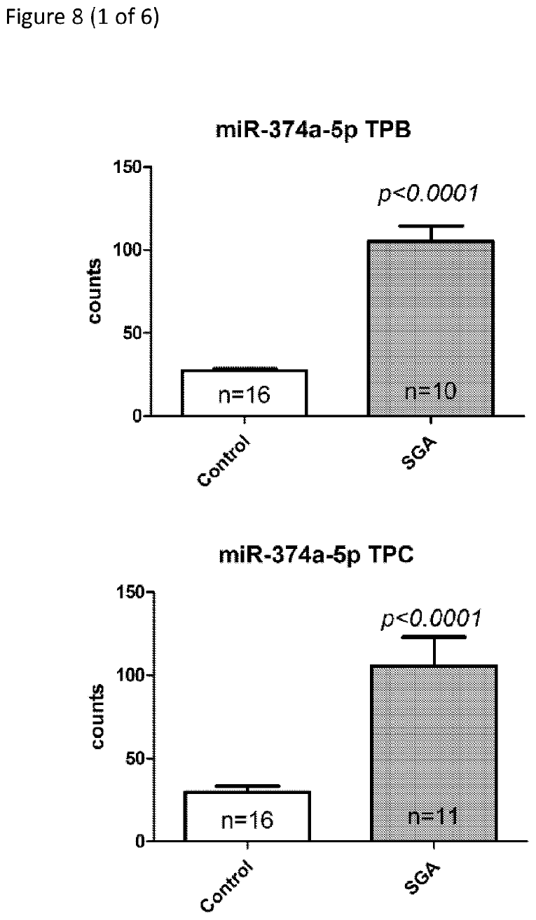

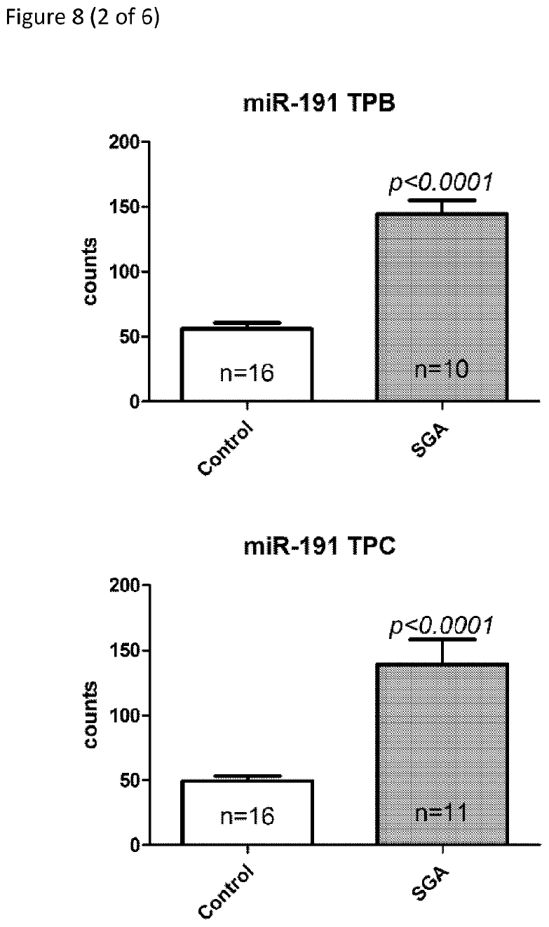

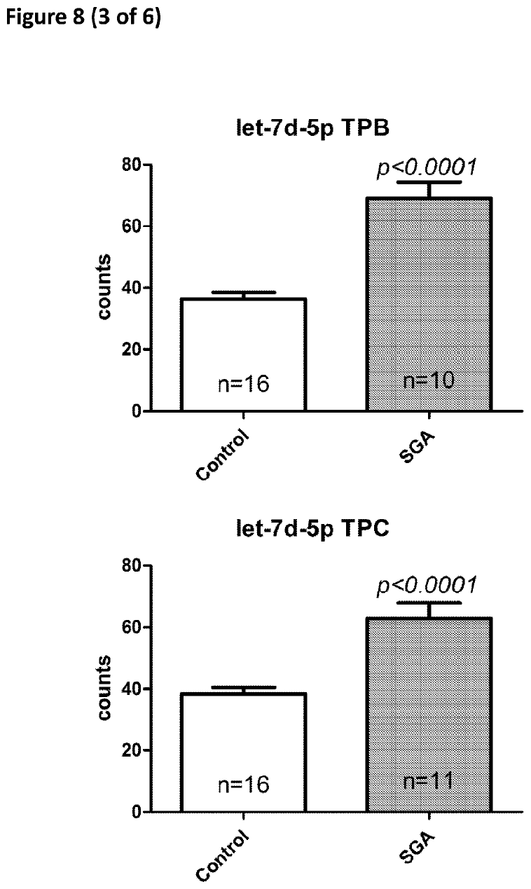

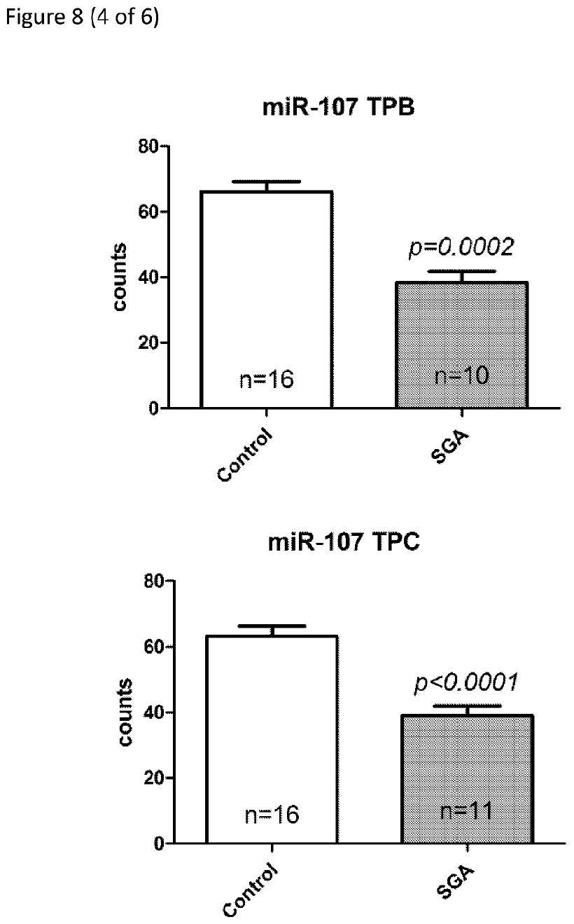

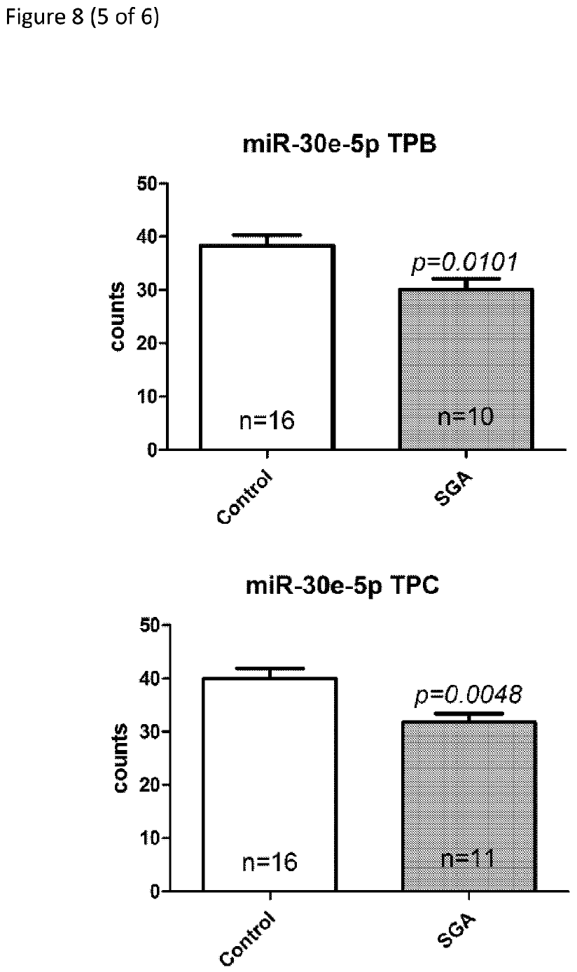

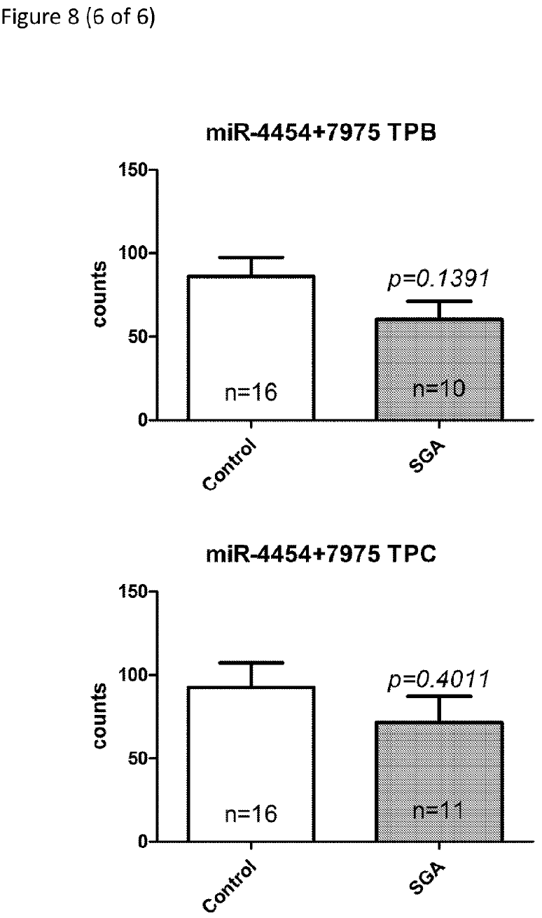

Univariate analysis of the nCounter probe counts for the 7 differentially expressed miRNAs were performed. At time-point A (12⁺⁰-14⁺⁶ weeks of gestation), miRNAs; hsa-miR-374a-5p, hsa-miR-191-5p, and hsa-let-7d-5p, were significantly increased in plasma from SGA cases (p<0.001 vs controls, Mann-Whitney U test; FIGS. 3a-c) and the remaining miRNAs hsa-miR-107, hsa-miR-30e-5p and hsa-miR-4454+miR-7975 were decreased in SGA cases compared to controls (p<0.001, p=0.0048, p=0.047 vs controls, Mann-Whitney U test; FIG. 3d-f). Similarly, the differential expression of these miRNA markers were observed at time-points B (15⁺⁰-17⁺⁶ weeks) and C (18⁺⁰-21⁺⁶ weeks) and there were no significant differences observed between the different time points (FIG. 8).

### Example 2

Technical Validation of nCounter miRNA Profiling Assay Data from Time-Point a Using Real-Time gPCR

For further validation, we focused on technically validating these candidate plasma miRNAs using real-time quantitative polymerase chain reaction (RT-qPCR). The normalisation of RT-qPCR data was performed to take the RNA extraction efficiency, reverse transcription efficiency and the endogenous miRNA differences into account. As there is a lack of consensus in endogenous miRNA controls for circulating miRNAs, the endogenous miRNA controls for this study were determined using the nCounter assay data. The combination of miRNAs with the least variation were identified using NormFinder algorithm (Andersen et al., 2004) and hsa-miR-30d-5p and hsa-let-7i-5p were used as controls.

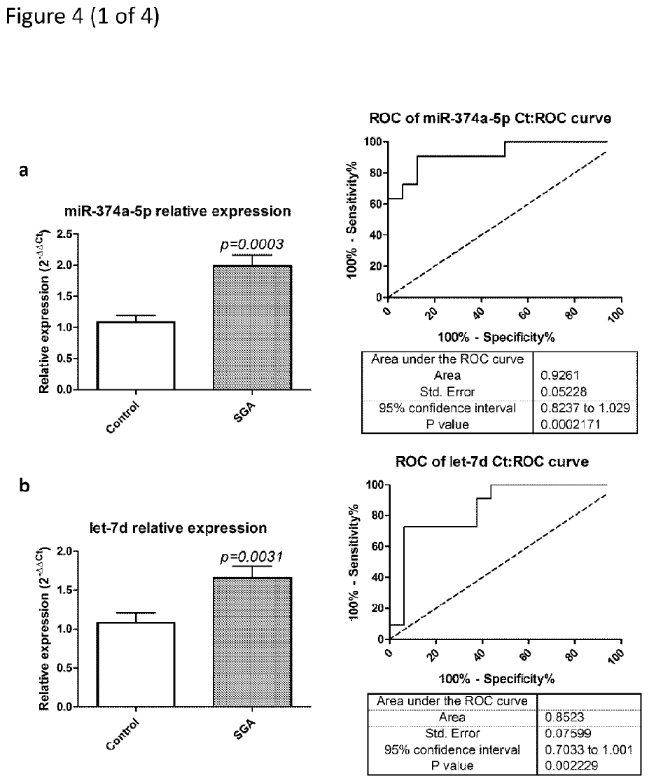

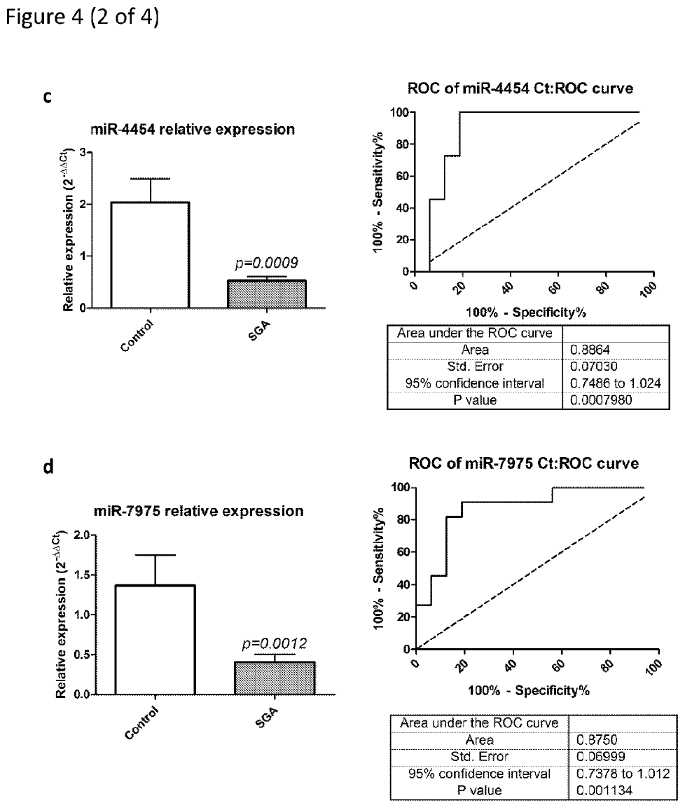

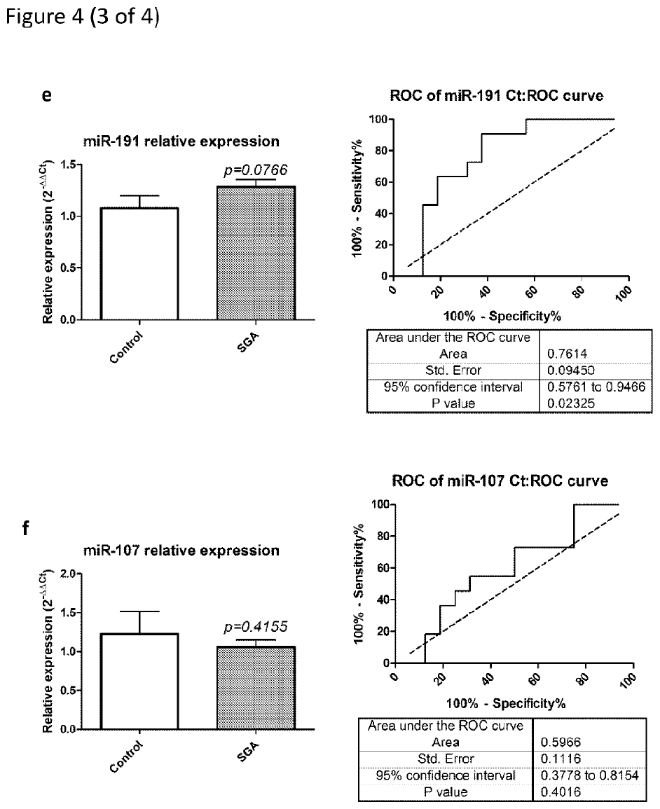

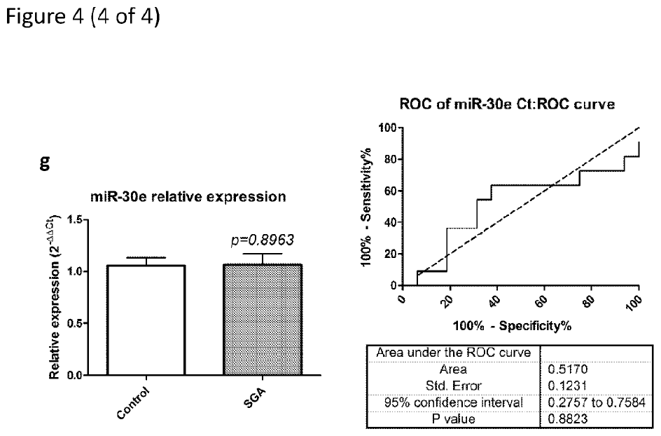

From 7 miRNAs identified using the nCounter assay, 4 miRNAs showed significant differences in expression between SGA cases and controls. These include hsa-miR-374a-5p (p=0.0003, Mann-Whitney U test), hsa-let-7d-5p (p=0.0031, Mann-Whitney U test), hsa-miR-4454 (p=0.0009, Mann-Whitney U test) and hsa-miR-7975 (p=0.0012, Mann-Whitney U test) (FIG. 4a-d). It was not possible to replicate the upregulation of hsa-miR-191 and the downregulation of hsa-miR-107 and hsa-miR-30e-5p in SGA cases from the nCounter assay data using RT-qPCR (FIG. 4e-g).

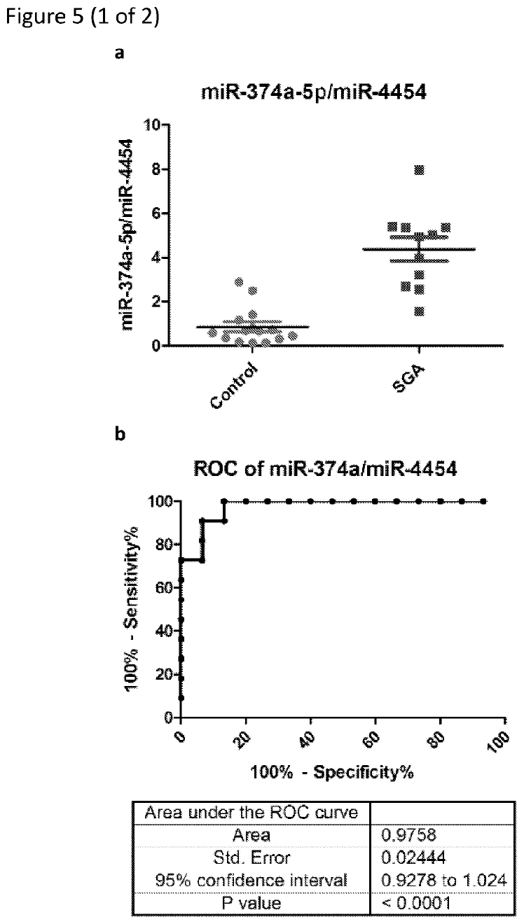

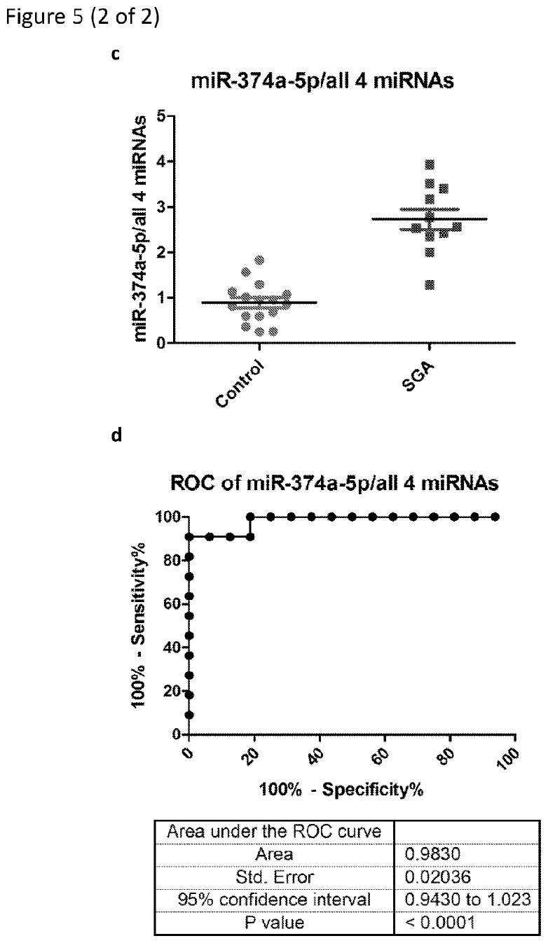

As hsa-miR-374a-5p and hsa-let-7d-5p were upregulated and hsa-miR-4454 and hsa-miR-7975 were downregulated in SGA cases compared to controls, we aimed to examine whether expression ratios constructed between these miRNA targets could improve their ability to differential SGA cases from controls. The expression ratios were calculated following the methods described in previous studies (Gordon et al., 2002, Avissar et al., 2009) where the relative expression of miRNAs that are upregulated in SGA cases (hsa-miR-374a-5p and hsa-let-7d-5p) were divided by those that are downregulated in SGA cases (hsa-miR-4454 and hsa-miR-7975). ROC analysis was performed to investigate the predictive ability of these miRNA expression ratios. The ratio of miR-374a-5p to miR-4454 demonstrated a strong predictive ability for SGA cases with AUC of 0.9758 (FIGS. 5a and b), and high sensitivity and specificity (AUC of 90.91 and 93.33, respectively). Similarly, the ratio of miR-374a-5p to the average of all 4 miRNAs that decrease in SGAs (miR-107, miR-30e-5p, miR-4454 and miR-7975) demonstrated AUC of 0.9830 (FIGS. 5c and d).

### Example 3

Discussion

One of the major challenges in modern medicine is the detection or diagnosis of a disease at an early stage. Early detection can vastly enhance the disease outcome as it allows administration of intervention at earlier phases of the disease. There has been a growing interest in miRNAs as potential biomarkers for the risk of pregnancy-related complications, including SGA babies. Since the discovery of detectable circulating miRNAs in biofluids, maternal biofluids such as serum and plasma have become an attractive sample choice for biomarker discovery. In this pilot study, we have identified seven miRNAs using nCounter miRNA profiling assay whose expressions in early pregnancy are associated with the risk of SGA births. Four of the seven identified miRNAs, hsa-miR-374a-5p, hsa-let-7d-5p, hsa-miR-4454 and hsa-miR-7975, were validated using RT-qPCR and the relative expression ratios were used to examine their ability to predict SGA births using ROC curve analysis. The hsa-miR-374a-5p to hsa-miR-4454 ratio exhibited the strongest predictive ability for SGAs with AUC >0.97.

FGR can be caused by fetal, placental or maternal factors including genetic abnormalities, dysfunctional trophoblastic invasion, hypoxia and inflammatory diseases (Lin & Santolaya-Forgas, 1998). There is limited evidence associating the four validated miRNA markers (hsa-miR-374a-5p, hsa-let-7d-5p, hsa-miR-4454 and hsa-miR-7975) with FGR and the functions of hsa-miR-7975 have not yet been studied. However, in s/i/co pathway analysis of the conserved target genes of these miRNAs using miRDIP (http://ophid.utoronto.ca/mirDIP) and pathDIP (http://ophid.utoronto.ca/pathDIP) demonstrated significant enrichment in various inflammatory/immune response- and cancer-related pathways (Table 3).

TABLE 3
Functional enrichment pathway analysis. The list of target
genes for the miRNA markers were identified using miRNA Data
Integration Portal (miRDIP; http://ophid.utoronto.ca/mirDIP),
which were used for pathway enrichment analysis by pathway Data
Integration Portal (pathDIP; http://ophid.utoronto.ca/pathDIP).
List of pathways enriched by the target genes (p < 0.01) of
differentially expressed miRNAs validated by RT-qPCR.
Functional enrichment pathway analysis

| Pathway name | p-value | FDR |
|---|---|---|
| MAPK signalling pathway | 9.35E−21 | 2.59E−18 |
| MicroRNAs in cancer | 9.81E−20 | 1.36E−17 |
| AMPK signalling pathway | 1.10E−08 | 1.80E−07 |
| TNF signalling pathway | 3.40E−08 | 4.10E−07 |
| Endocytosis | 5.99E−07 | 5.73E−06 |
| T cell receptor signalling pathway | 1.72E−06 | 1.36E−05 |
| Cytokine-cytokine receptor interaction | 1.78E−05 | 9.13E−05 |
| Chemokine signaling pathway | 4.04E−05 | 1.90E−04 |
| Platelet activation | 4.31E−05 | 1.93E−04 |
| Fc gamma R-mediated phagocytosis | 7.20E−05 | 3.07E−04 |

The top enriched pathways includes mitogen-activated protein kinase (MAPK) signalling, miRNAs in cancer, tumour necrosis factor (TNF) signalling, cytokine-cytokine receptor interaction as well as chemokine signalling. Chemokines such as CCL5 is found to be upregulated in first trimester trophoblasts (Critchley et al., 1999) where it plays a role in the activation and recruitment of immune cells in the developing placenta to drive post-implantation tissue remodelling (Abrahams et al., 2004). It has been reported that increase in hsa-miR-374a-5p suppresses CCL5 expression (Cross et al., 2015) and thus potentially impairing the process of placental development. Moreover, hsa-miR-4454 has been shown to be involved in the regulation of matrix metalloproteinase (MMP) expression (Nakamura et al., 2016). The fine-tuning of MMP activity, which is directly proportional to its expression, in the maternal-fetal interface is important for the successful outcome for trophoblast invasion, implantation as well as placentation (Zhu et al., 2012). Increase in the levels of hsa-let-7d-5p has been associated with preeclampsia and the expression of MMPs (Dai & Cai, 2018) as well as chemokines such as CCL7 (Su et al, 2014). Therefore, the validated miRNA markers have a potential biological role in the setting of FGR and SGA births.

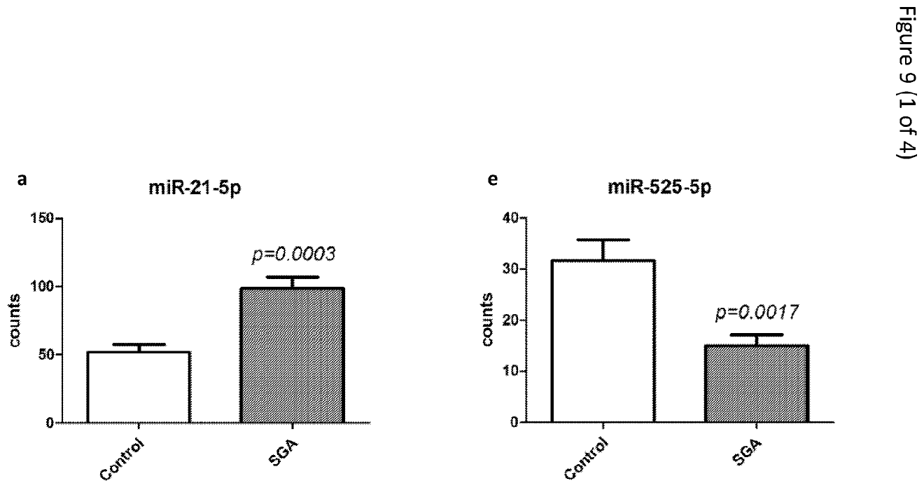

Previous studies on the discovery of miRNA biomarkers for the prediction of SGA births have been mainly focused on the changes in the expression of placental miRNAs which directly reflect dysfunctional placenta (Cindrova-Davies et al., 2013, Hromadnikova et al, 2015a, Guo et al., 2013, Huang et al, 2013). However, some of the differential expression of placental miRNAs were not detected in maternal circulation. Higasijima et al have reported significantly lower expression of hsa-miR-518b, miR1323, miR-520h and miR-519d in the placenta of FGR pregnancies, however, the expression of these placental miRNAs were not reflected in the maternal plasma (Higashijima et al., 2013). Similar discrepancies in the differential expression of placental miRNAs in placental tissue and maternal plasma were reported by Hromadnikova et al. In 2012, they found no significant differences in the expression of placental miRNAs, miR-517, miR-510a and miR-525, in maternal plasma of FGR cases and controls (Hromadnikova et al, 2012), but their work in 2015 demonstrated downregulation of these miRNAs in placental tissue of FGR cases compared to controls (Hromadnikova et al., 2015b). Although the expression of placental miRNAs were not included in our top 50 highly expressed miRNAs from nCounter assay, we did observe differential expression of miR-21-5p, miR-14-5p, miR-574-3p, miR-518b and miR-525-5p (FIG. 9a-e), complementary to previously published data (Cindrova-Davies et al., 2013, Hromadnikova et al., 2015a, Guo et al., 2013, Higashijima et al., 2013, Hromadnikova et al., 2015b). In contrast to our nCounter assay data where the placental miRNAs, miR-141-3p, miR-499a-5p and miR-424-5p, were found to be decreased in SGA cases (FIG. 9f-h), other studies demonstrated an increase in their expression in FGR cases compared to healthy controls (Hromadnikova et al., 2015a, Guo et al, 2013, Tang et al., 2013). Due to the inconsistent findings in the detection of placenta-specific miRNAs in maternal circulation, these results should be taken in with caution and further investigations into understanding the processes involved in the release of placenta-specific miRNAs to the maternal circulation are required.

In conclusion, we have identified 7 differentially expressed plasma miRNAs which contribute to the discrimination of SGA cases and normal controls using Nanostring nCounter miRNA profiling assay. Four of these 7 miRNAs were technically validated at 12-14⁺⁶ weeks gestation using RT-qPCR where two were elevated and two were decreased in maternal plasma in women who went on to delivery SGA babies. The expression ratios of hsa-miR-374a-5p to hsa-let-7d-5p demonstrated the ability to predict SGA status. Furthermore, we confirmed previous findings of differentially expressed placental miRNAs in SGA cases.

### Example 4

Methods

Recruitment of Participants and Sample Collection

The Hertfordshire Research Ethics Committee approved the study protocol (22/02/2011-REC reference number 11/H0311/6) and informed written consents were received from each participant.

The participants were women with singleton pregnancies attending antenatal clinics at Imperial College Healthcare NHS Trust Hospitals in London, UK. Women with pre-existing diseases and those who developed obstetric complications, such as pre-eclampsia, gestational diabetes, and obstetric cholestasis, were excluded from the study. Maternal plasma samples were collected prospectively at three time-points in mid-pregnancy; 12⁺⁰-14⁺⁶ (time-point A), 15⁺⁰-17⁺⁶ (time-point B) and 18⁺⁰-21⁺⁶ (time-point C) weeks gestation. The plasma samples were extracted from whole blood collected from participants. The blood was kept on ice and processed within 30 min of collection by centrifugation at 1300×g for 10 min at 4° C. Isolated plasma were stored in aliquots at −80° C. until further analysis. Samples demonstrating macroscopic haemolysis were discarded.

Following delivery, the samples were divided into SGA cases, defined as birth-weight below the 5^{th} centile for gestational age and ethnicity, and controls with birth-weights appropriate for gestational age (10^{th}-90^{th} centile) (Gardosi et al., 1992). Table 1 shows both demographic and clinical characteristics of the study cohort.

RNA Extraction

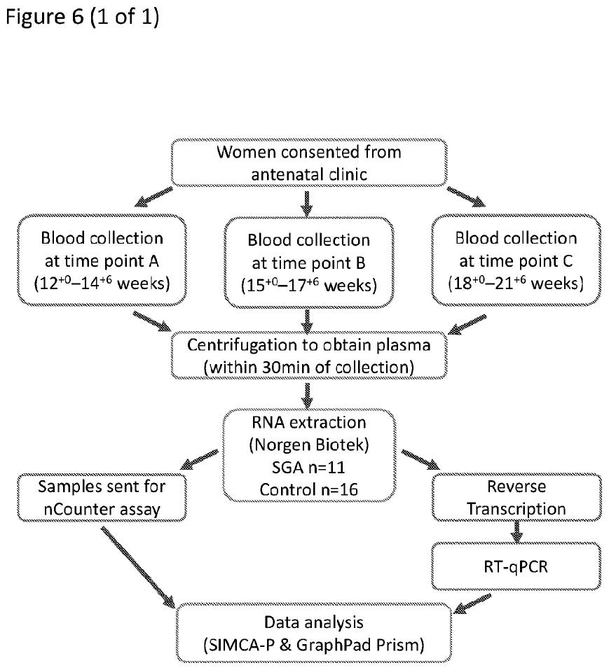

A flowchart for the collection and extraction of miRNAs from plasma is shown in FIG. 6. Plasma aliquots were placed on ice to thaw completely prior to centrifugation at 800×g for 10 min at 4° C. Only the upper 750 μl of plasma was used for the RNA extraction to minimise cellular and platelet contamination (Cheng et al., 2013). RNAs were obtained with the Plasma/Serum Circulating and Exosomal RNA Purification Mini Kit (Norgen Biotek, Ontario, Canada) according to the manufacturer's recommendations. In accordance with the protocol, 5000 attomoles synthetic cel-miR-254 (Integrated DNA technologies BVBA, Leuven, Belgium) was added to plasma following lysis and denaturation as a spike-in control for normalisation of any technical variation that may have occurred during the extraction process. The eluted RNA was further purified using the Amicon Ultra YM-3 columns (Merck Millipore, Darmstadt, Germany).

nCounter miRNA Assay and Data Analysis

Extracted RNAs were subjected to nCounter™ plasma miRNA profiling (Nanostring, Seattle, USA) which allows direct assessment of 800 human miRNA target expression levels without cDNA synthesis or enzymatic reactions (Geiss et al., 2008). The resulting counts were analysed by the nCounter Digital Analyser. The raw counts from nCounter were normalised to the expression of top 100 expressed miRNAs. Only those miRNAs expressed above background level in greater than 50% of samples from any outcome group were used for further analysis, where background level was defined by 2-standard deviations above the mean negative control counts. Top 50 highly expressed miRNAs were used for further analysis.

Multivariate analyses were carried out using SIMCA-P version 13.0.2 software (soft independent modelling of class analogies-P, Umetrics, Umeå, Sweden). Principal component analysis (PCA) was first performed comparing SGA cases to controls. Following this, a supervised partial least-squares discriminant analysis (PLS-DA) was performed to identify clustering of groups. The validity of PLS-DA model was assessed by response permutation testing (100 repetitions). For data which are not over-modelled, the R² (correlation) and Q² (predictability) coefficients should degrade to <0.3 and <0, respectively (Beyoglu et al., 2013). A list of plasma miRNAs that are differentially expressed in SGA cases compared to controls was identified based on variable importance for projection (VIP) score of >1 in the PLS-DA model. Top 7 miRNA markers identified were subjected to univariate analysis using GraphPad Prism 5 (GraphPad Software, CA, USA) those with ρ-value of <0.05 in the Mann-Whitney U tests were considered significantly different. Hierarchical clustering heat map was generated based on Ward's method (Ward Jr, J. H., Hierarchical Grouping to Optimize an Objective Function. Journal of the American Statistical Association, 1963. 58(301): p. 236-244) ( ) using ClustVis (https://biit.cs.ut.ee/clustvis/) (Metsalu & Vilo, 2015).

Real-Time Quantitative Polymerase Chain Reaction (RT-qPCR) and Data Analysis

RT-qPCR was used to technically validate the nCounter miRNA assay data. RNA extracted from plasma were reverse transcribed to cDNA using miRCURY LNA™ Universal RT miRNA cDNA synthesis kit II (Exiqon, Vedbaek, Denmark) with the addition of 0.625 μl miRNA UniSp6 (10⁸ copies/μl) to allow normalisation of variation that may occur during the reverse transcription reaction. 2 μl RNA which corresponds to the mass of RNA derived from ≈60 μl of starting plasma was used for the 10 μl reverse transcription reaction following manufacturer's instructions.

RT-qPCR was performed using custom pick and mix manels with LNA™ primers (Exiqon, Vedbaek, Denmark). The ExiLENT SYBR® Green master mix (Exiqon, Vedbaek, Denmark) was used as per manufacturer's protocols and the reactions were carried out on ABI StepOnePlus (Life Technologies, Paisley, UK). The cycle conditions for miRNA targets included initial polymerase activation/denaturation step at 95° C. for 10 min, followed by 45 amplification cycles consisting of 95° C. for 10 sec and 60° C. for 1 min. Melt curve analyses were used to confirm single PCR product. Raw fluorescence data were collected by the StepOne software v2.3 (Life Technologies, Paisley, UK) and exported for further analyses.

The LinRegPCR program v2017.1 (Ruijter et al., 2009) was used to determine Cq values and amplification efficiencies for all samples and miRNA targets. Pre-processing of Cq data consisted of normalisation to the interplate calibrator, RNA extraction control, cel-miR-254, and the reverse transcription control, UniSp6. In order to evaluate the miRNA expression, Cq values from LinRegPCR were normalised to two endogenous controls and fold differences to control samples were obtained (2^{-ΔCq}).

The two endogenous miRNA controls were identified from the nCounter assay data using NormFinder (Andersen et al., 2004). Normfinder incorporates the inter- and intra-group variances and calculates the stability value per miRNA target as a measure of expression stability. It also identifies the best combination of miRNA targets to provide the highest stability. Statistical analyses were performed using GraphPad Prism 5 (GraphPad Software, CA, USA) to compare ΔCq values obtained from SGA cases and controls. Non-parametric Mann-Whitney U-test (2-tailed) was used with significance defined as ρ-value of <0.05. Receiver operating characteristic (ROC) curves for miRNA expression ratios were generated by GraphPad Prism 5 (GraphPad Software, CA, USA).

### Example 5

Introduction

We have previously identified 7 differentially expressed circulating miRNAs predictive of SGA in a discovery study cohort of pregnant women. Here, we aimed to validate these findings in an independent patient cohort sampled between 12-14⁺⁶ weeks gestation.

Methods

Participant Recruitment and Sample Collection

Ethical approval was granted by the Hertfordshire Research Ethics Committee (22/02/2011-REC reference number 11/H0311/6) and informed written consent was received from all participants. All research was performed in accordance with the relevant guidelines and regulations. Participants were women with singleton pregnancies attending antenatal clinics at Imperial College Healthcare NHS Trust Hospitals in London, UK. Women with pre-existing diseases and those who developed obstetric complications, such as pre-eclampsia, gestational diabetes, and obstetric cholestasis, were excluded from the study. Maternal plasma samples were extracted from whole blood collected prospectively at 12-14⁺⁶ weeks gestation. Blood was kept on ice and processed for plasma within 30 min of collection by centrifugation at 1300×g for 10 min at 4° C. Isolated plasma were stored in aliquots at −80° C. until further analysis. Following delivery, the samples were divided into SGA cases, defined as birth-weight below the 5th centile for gestational age and ethnicity, and controls with birth-weights appropriate for gestational age (44).

RNA Extraction

Plasma aliquots stored at −80° C. were thawed on ice prior to centrifugation at 800×g for 10 min at 4° C. The upper 750 μl of plasma was used for the RNA extraction to minimize cellular and platelet contamination (45). RNAs were isolated with the Plasma/Serum Circulating and Exosomal RNA Purification Mini Kit (Norgen Biotek) according to the manufacturer's recommendations. To allow normalization of any technical variation that may occur during the RNA extraction process, 5000 attomoles of cel-miR-254 (Integrated DNA technologies BVBA) was added to plasma following lysis and denaturation as a spike-in control. The eluted RNA was concentrated and further purified using the Amicon Ultra YM-3 columns (Merck Millipore).

RT-qPCR and Data Analysis

RNA extracted from plasma were reverse transcribed to cDNA using miRCURY LNA™ Universal RT miRNA cDNA synthesis kit (Qiagen) with the addition of UniSp6 (10⁸ copies/μl) to allow for normalization of variation that may occur during the reverse transcription process. A total of 2 μl RNA, corresponding to the mass of RNA derived from approximately 60 μl of starting plasma, was used for the reverse transcription reaction following manufacturer's instructions.

RT-qPCR was performed using custom pick and mix panels with LNA™ primers (Qiagen). The ExiLENT SYBR® Green master mix (Qiagen) was used as per manufacturer's instructions and the reactions were carried out on an ABI StepOnePlus Real Time PCR System (Life Technologies). The cycle conditions for miRNA targets included initial polymerase activation/denaturation step at 95° C. for 10 min, followed by 45 amplification cycles consisting of 95° C. for 10 sec and 60° C. for 1 min. Melt curve analyses were used to confirm a single PCR product. Raw fluorescence data were collected by the StepOne software v2.3 (Life Technologies) and exported for further analyses.

The LinRegPCR program v2017.1 (49) was used to determine Cq values and amplification efficiencies for all samples and miRNA targets. Pre-processing of Cq data consisted of normalization to the interplate calibrator, RNA extraction control, cel-miR-254, and the reverse transcription control, UniSp6. In order to evaluate the miRNA expression, Cq values from LinRegPCR were normalized to two endogenous controls identified using NormFinder (52) and fold differences to control samples were obtained (2^{-ΔCq}). ΔCq values obtained from SGA cases and controls were subsequently used for univariate analyses with a non-parametric Mann-Whitney U-test (2-tailed) where a ρ-value of <0.05 was considered to be statistically significant. Receiver operating characteristic (ROC) curves for miRNA expression ratios were generated using GraphPad Prism 5 (GraphPad Software), and ROC curves using multiple predictors were generated using SPSS 25 (Statistical Package for the Social Sciences). Relative expressions (2^{-ΔCq}) are plotted as mean±SEM.

Results

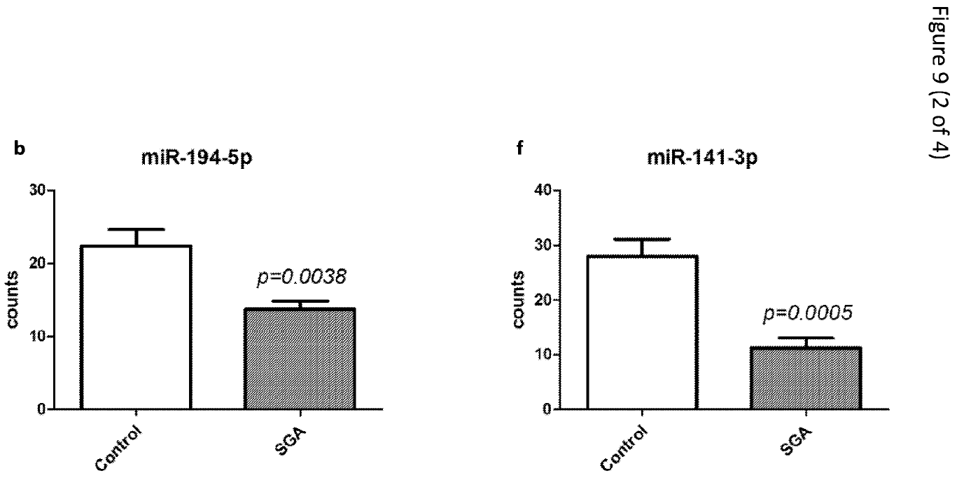

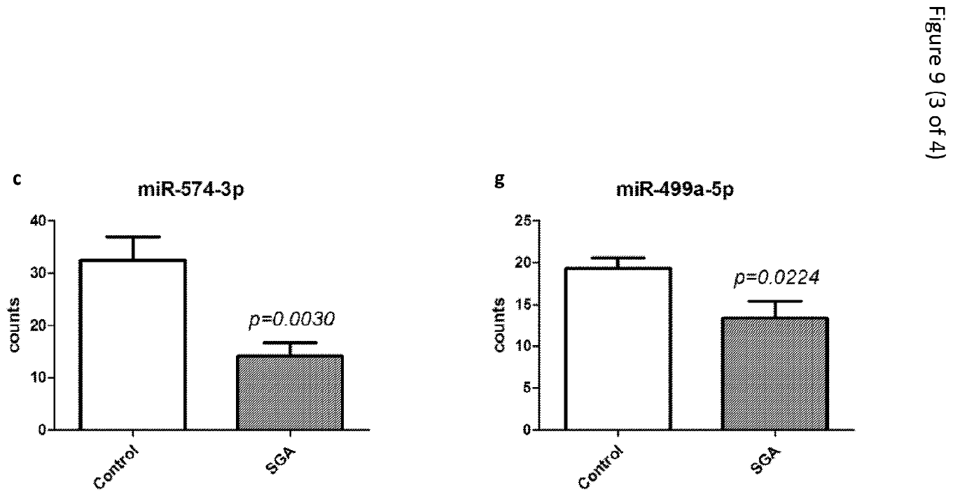

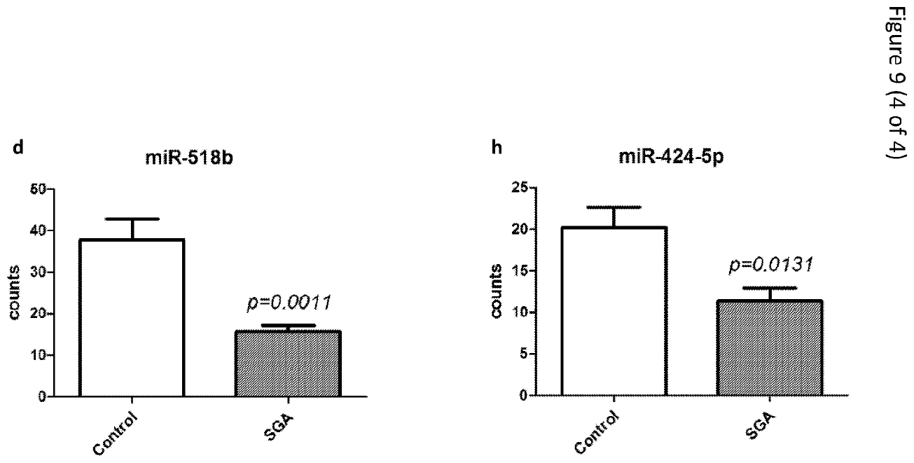

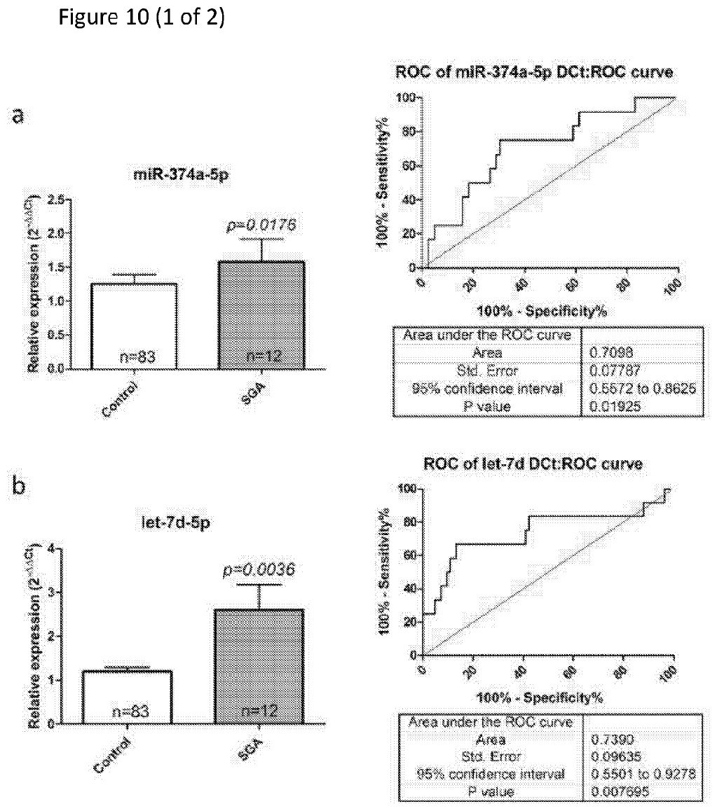

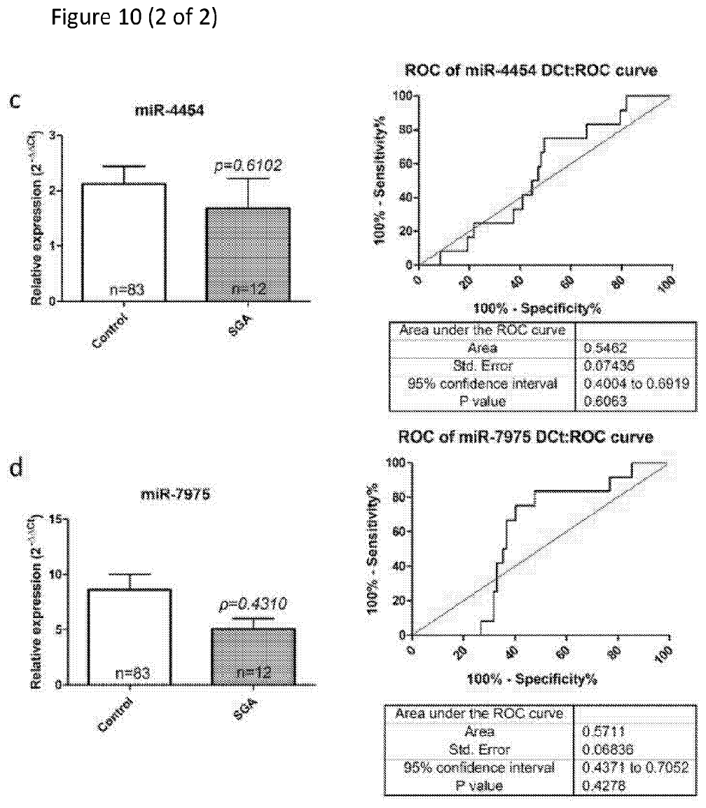

From a total of 95 participants included in the study, 12 had SGA and 87 had normal weight babies. The demographic and clinical characteristics of both groups were similar with the exception of birthweight (p<0.0001), as expected (Table 4). From 7 identified miRNA markers, 4 miRNAs hsa-miR-374a-5p, hsa-let-7d-5p, hsa-miR-4454 and hsa-miR-7975 were validated using RT-qPCR. Consistent with our previous findings, significantly higher expression of hsa-miR-374a-5p and hsa-let-7d-5p were observed in plasma from women who subsequently delivered SGA babies (FIGS. 10a and b). These miRNAs individually demonstrated good predictive ability for SGA cases with AUC greater than 0.70 (FIGS. 10a and b). Consistently lower expression of hsa-miR-4454 and hsa-miR-7975 were observed in SGA cases compared to normal controls (FIGS. 10c and d).

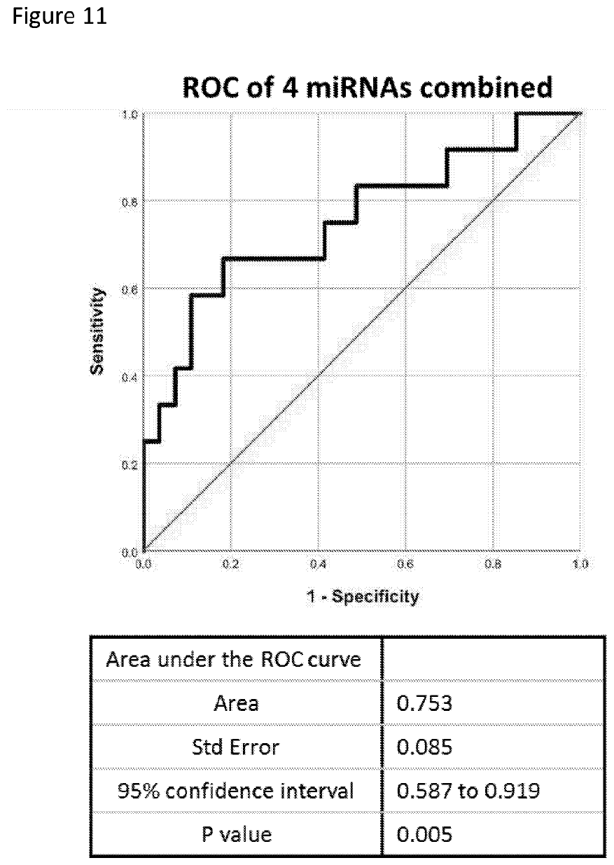

The replication of the earlier findings in an independent population of women supports our hypothesis that specific miRNAs may act as peripherally available biomarkers of future SGA deliveries. The predictive ability of these miRNA markers were enhanced by combining the relative expression levels of all 4 validated miRNAs. Using the relative expressions of hsa-miR-374a-5p, hsa-let-7d-5p, hsa-miR-4454 and hsa-miR-7975 improved the predictive ability for SGA cases, with the AUC of 0.75 (FIG. 11).

TABLE 4
Demographic and Clinical variables.

| | | Controls | | SGA | | p- |
|---|---|---|---|---|---|---|
| | | (n = 83) | | (n = 12) | | value* |
| Maternal age (SD) | | 33 | (4.35) | 35 | (3.89) | 0.1979 |
| Ethnicity (%) | Caucasian | 53 | (63.9) | 8 | (66.7) | 0.8026 |
| | Asian | 20 | (24.1) | 2 | (16.7) | |
| | Black | 10 | (12) | 2 | (16.7) | |

| | | | | | |
|---|---|---|---|---|---|
| Gestational age | 38.7 | (1.79) | 39.2 | (1.18) | 0.4324 |
| at delivery (SD) | | | | | |

| | | | | |
|---|---|---|---|---|
| Baby gender | Male | 50 | 5 | 0.3485 |
| (%) | Female | 33 | 7 | |

| | | | | | |
|---|---|---|---|---|---|
| Birthweight (SD) | 3524.3 | (497.3) | 2548 | (206.6) | <0.0001 |

*p-value corresponds to Mann-Whitney U test (continuous variables) or Chi-squared test (categorical variables) for the difference in study participants' characteristics between control and SGA groups.

### REFERENCES

1. Fitzhardinge, P. M. and E. M. Steven, The small-for-date infant. II. Neurological and intellectual sequelae. Pediatrics, 1972. 50(1): p. 50-7.
2. Low, J. A., et al., Intrauterine growth retardation: a preliminary report of long-term morbidity. Am J Obstet Gynecol, 1978. 130(5): p. 534-45.
3. Barker, D. J., et al., Weight in infancy and death from ischaemic heart disease. Lancet, 1989. 2(8663): p. 577-80.
4. Phipps, K., et al., Fetal growth and impaired glucose tolerance in men and women. Diabetologia, 1993. 36(3): p. 225-8.
5. Hadlock, F. P., R. L. Deter, and R. B. Harrist, Sonographic detection of abnormal fetal growth patterns. Clin Obstet Gynecol, 1984. 27(2): p. 342-51.
6. Harding, K., S. Evans, and J. Newnham, Screening for the small fetus: a study of the relative efficacies of ultrasound biometry and symphysiofundal height. Aust N Z J Obstet Gynaecol, 1995. 35(2): p. 160-4.
7. Lee, A. C., et al., National and regional estimates of term and preterm babies born 5 small for gestational age in 138 low-income and middle-income countries in 2010. Lancet Glob Health, 2013. 1(1): p. e26-36.
8. Black, R. E., Global Prevalence of Small for Gestational Age Births. Nestle Nutr Inst Workshop Ser, 2015. 81: p. 1-7.
9. Wright, J., Morse, K., Kady, S. and Francis, A., Audit of fundal height measurement plotted on customised growth charts. MIDIRS Midwifery Digest, 2006. 16: p. 341-345.
10. Bartel, D. P., MicroRNAs: genomics, biogenesis, mechanism, and function. Cell, 2004. 116(2): p. 281-97.
11. Sonkoly, E. and A. Pivarcsi, microRNAs in inflammation. Int Rev Immunol, 2009. 28(6): p. 535-61.
12. Hwang, H. W. and J. T. Mendell, MicroRNAs in cell proliferation, cell death, and tumorigenesis. Br J Cancer, 2006. 94(6): p. 776-80.
13. Jovanovic, M. and M. O. Hengartner, miRVAs and apoptosis: RNAs to die for. Oncogene, 2006. 25(46): p. 6176-87.
14. Wu, L., et al., Circulating microRNAs are elevated in plasma from severe preeclamptic pregnancies. Reproduction, 2012. 143(3): p. 389-97.
15. Zhao, Z., et al., Circulating microRNA miR-323-3p as a biomarker of ectopic pregnancy. Clin Chem, 2012. 58(5): p. 896-905.
16. Zhao, C., et al., Early second-trimester serum miRNA profiling predicts gestational diabetes mellitus. PLoS One, 2011. 6(8): p. e23925.
17. Hu, Y., et al., Two common SNPs in pri-miR-125a alter the mature miRNA expression and associate with recurrent pregnancy loss in a Han-Chinese population. RNA Biol, 2011. 8(5): p. 861-72.
18. Gray, C., et al., Maternal plasma miRNAs as biomarkers during mid-pregnancy to predict later spontaneous preterm birth: a pilot study. Sci Rep, 2017. 7(1): p. 815.
19. Winger, E. E., J. L. Reed, and X. Ji, Early first trimester peripheral blood cell microRNA predicts risk of preterm delivery in pregnant women: Proof of concept. PLoS One, 2017. 12(7): p. e0180124.
20. Maccani, M. A., J. F. Padbury, and C. J. Marsit, miR-16 and miR-21 expression in the placenta is associated with fetal growth. PLoS One, 2011. 6(6): p. e21210.
21. Rodosthenous, R. S., et al., Second trimester extracellular microRNAs in maternal blood and fetal growth: An exploratory study. Epigenetics, 2017. 12(9): p. 804-810.
22. Tam, S., et al., Robust global microRNA expression profiling using next-generation sequencing technologies. Lab Invest, 2014. 94(3): p. 350-8.
23. Atherton, H. J., et al., Metabolomics of the interaction between PPAR-alpha and age in the PPAR-alpha-null mouse. Mol Syst Biol, 2009. 5: p. 259.
24. Semmo, N., et al., Metabolomics reveals that aldose reductase activity due to AKR1B10 is upregulated in hepatitis C virus infection. J Viral Hepat, 2015. 22(7): p. 617-24.
25. Andersen, C. L., J. L. Jensen, and T. F. Orntoft, Normalization of real-time quantitative reverse transcription-PCR data: a model-based variance estimation approach to identify genes suited for normalization, applied to bladder and colon cancer data sets. Cancer Res, 2004. 64(15): p. 5245-50.
26. Gordon, G. J., et al., Translation of microarray data into clinically relevant cancer diagnostic tests using gene expression ratios in lung cancer and mesothelioma. Cancer Res, 2002. 62(17): p. 4963-7.
27. Avissar, M., et al., MicroRNA expression ratio is predictive of head and neck squamous cell carcinoma. Clin Cancer Res, 2009. 15(8): p. 2850-5.
28. Lin, C. C. and J. Santolaya-Forgas, Current concepts of fetal growth restriction: part l. Causes, classification, and pathophysiology. Obstet Gynecol, 1998. 92(6): p. 1044-55.
29. Critchley, H. O., et al., Role of inflammatory mediators in human endometrium during progesterone withdrawal and early pregnancy.J Clin Endocrinol Metab, 1999. 84(1): p. 240-8.
30. Abrahams, V. M., et al., Macrophages and apoptotic cell clearance during pregnancy. Am J Reprod Immunol, 2004. 51(4): p. 275-82.
31. Cross, C. E., et al., Oxidative Stress Alters miRNA and Gene Expression Profiles in Villous First Trimester Trophoblasts. Biomed Res Int, 2015. 2015: p. 257090.
32. Nakamura, A., et al., Identification of microRNA-181a-5p and microRNA-4454 as mediators of facet cartilage degeneration. JCI Insight, 2016. 1(12): p. e86820.
33. Zhu, J. Y., Z. J. Pang, and Y. H. Yu, Regulation of trophoblast invasion: the role of matrix metalloproteinases. Rev Obstet Gynecol, 2012. 5(3-4): p. e137-43.
34. Dai, X. and Y. Cai, Down-regulation of microRNA let-7d inhibits the proliferation and invasion of trophoblast cells in preeclampsia. J Cell Biochem, 2018. 119(1): p. 1141-1151.
35. Su, B., et al., Let-7d suppresses growth, metastasis, and tumor macrophage infiltration in renal cell carcinoma by targeting COL3A1 and CCL7. Mol Cancer, 2014. 13: p. 206.
36. Cindrova-Davies, T., et al., Reduced cystathionine gamma-lyase and increased miR-21 expression are associated with increased vascular resistance in growth-restricted pregnancies: hydrogen sulfide as a placental vasodilator. Am J Pathol, 2013. 182(4): p. 1448-58.
37. Hronadnikova, l., et al., Cardiovascular and Cerebrovascular Disease Associated microRNAs Are Dysregulated in Placental Tissues Affected with Gestational Hypertension, Preeclampsia and Intrauterine Growth Restriction. PLoS One, 2015a. 10(9): p. e0138383.
38. Guo, L., et al., Differentially expressed microRNAs and affected biological pathways revealed by modulated modularity clustering (MMC) analysis of human preeclamptic and IUGR placentas. Placenta, 2013. 34(7): p. 599-605.
39. Huang, L., et al., Increased levels of microRNA-424 are associated with the pathogenesis of fetal growth restriction. Placenta, 2013. 34(7): p. 624-7.
40. Higashijima, A., et al., Characterization of placenta-specific microRNAs in fetal growth restriction pregnancy. Prenat Diagn, 2013. 33(3): p. 214-22.
41. Hromadnikova, l., et al., Absolute and relative quantification of placenta-specific micromas in maternal circulation with placental insufficiency-related complications. J Mol Diagn, 2012. 14(2): p. 160-7.
42, Hromadnikova, l., et al., Expression profile of C19MC microRNAs in placental tissue in pregnancy-related complications. DNA Cell Biol, 2015b. 34(6): p. 437-57.
43. Tang, Q., et al., miR-141 contributes to fetal growth restriction by regulating PLAG1 expression. PLoS One, 2013. 8(3): p. e58737.
44. Gardosi, J., et al., Customised antenatal growth charts. Lancet, 1992. 339(8788): p. 283-7.
45. Cheng, H. H., et al., Plasma processing conditions substantially influence circulating microRNA biomarker levels. PLoS One, 2013. 8(6): p. e64795.
46. Geiss, G. K., et al., Direct multiplexed measurement of gene expression with color-coded probe pairs. Nat Biotechnol, 2008. 26(3): p. 317-25.
47. Beyoglu, D., et al., Tissue metabolomics of hepatocellular carcinoma: tumor energy metabolism and the role of transcriptomic classification. Hepatology, 2013. 58(1): p. 229-38.
48. Metsalu, T. and J. Vilo, ClustVis: a web tool for visualizing clustering of multivariate data using Principal Component Analysis and heatmap. Nucleic Acids Res, 2015. 43(W1): p. W566-70.
49. Ruijter, J. M., et al., Amplification efficiency: linking baseline and bias in the analysis of quantitative PCR data. Nucleic Acids Res, 2009. 37(6): p. e45.
50. Dobson P C, Abell D A, and Beischer N A (1981) Mortality and Morbidity of Fetal Growth Retardation. ANZJOG, 21(2), p 69-72.
51. Ward Jr, J. H., Hierarchical Grouping to Optimize an Objective Function. Journal of the American Statistical Association, 1963. 58(301): p. 236-244
52. Andersen C L, Jensen J L, Orntoft T F. Normalization of real-time quantitative reverse transcription-PCR data: a model-based variance estimation approach to identify genes suited for normalization, applied to bladder and colon cancer data sets. Cancer research, 2004. 64(15): 5245-50.

1. A method for predicting if a baby will be born at a birth-weight that is less than the 5^{th} centile for the particular gestational age at birth, wherein the method comprises determining the absolute level, amount or concentration or the relative level, amount or concentration of any one or more of the following miRNAs in a test sample obtained from the mother:
hsa-miR-374a-5p, hsa-let-7d-5p, hsa-miR-4454, hsa-miR-7975, hsa-miR-191, hsa-miR-107, or hsa-miR-30e-5p.

2. The method according to claim 1 wherein the method comprises determining the amount of:
a) hsa-miR-374a-5p;
b) hsa-let-7d-5p;
c) hsa-miR-374a-5p or hsa-let-7d-5p;
d) hsa-miR-374a-5p and hsa-miR-4454;
e) hsa-miR-374a-5p, hsa-let-7d-5p, hsa-miR-4454, and hsa-miR-7975;
f) has-miR-374a-5p, hsa-miR-4454, hsa-miR-7975, hsa-miR-107 and hsa-miR-30e-5p; or
g) hsa-miR-374a-5p, hsa-let-7d-5p, hsa-miR-4454, hsa-miR-7975, hsa-miR-191, hsa-miR-107, and hsa-miR-30e-5p.

3. The method according to claim 1 wherein the method comprises determining the ratio of any 1, 2, 3, 4, 5, 6, or 7 of the relative or absolute amounts of any of the hsa-miR-374a-5p, hsa-let-7d-5p, hsa-miR-191-5p, hsa-miR-4454, hsa-miR-7975, hsa-miR-107, and hsa-miR-30e-5p miRNAs is calculated, optionally
i) determining the ratio of the relative or absolute amounts of hsa-miR-374a-5p to hsa-miR-4454; and/or
ii) determining the ratio of the relative expression or absolute amounts of hsa-miR-374a to the average relative expression or absolute amounts of all 4 miRNAs that decrease in SGA cases (hsa-miR-30e, hsa-miR-107, hsa-miR-4454 and hsa-miR-7975).

4. The method of claim 1 wherein the amount of the miRNA(s) determined in the sample obtained from the mother, or the ratio of claim 3, is compared to the amount of the same miRNA(s) or the ratio in a control sample or control samples, or is compared to a control value or control values.

5. The method of claim 4 wherein the control sample or control samples is a sample that was:
a) taken from a pregnant control subject who later gave birth to a baby that is an appropriately grown baby (AGA), optionally wherein the control sample was taken from the control subject at substantially the same gestation or stage in pregnancy as the gestation or stage at which the test sample is taken from the mother; and/or
b) taken from a pregnant control subject who later gave birth to a baby that is SGA, optionally wherein the control sample was taken from the control subject at substantially the same gestation or stage in pregnancy as the gestation or stage at which the test sample is taken from the mother.

6. (canceled)

7. The method of claim 4 wherein the control sample is a sample that was taken from the mother at an earlier stage in pregnancy, or wherein the control value is the average level, amount or concentration of the miRNA(s) determined from a number of control samples that were
a) taken from a pregnant control subject who later gave birth to a baby that is an appropriately grown baby (AGA), optionally wherein the control sample was taken from the control subject at substantially the same gestation or stage in pregnancy as the gestation or stage at which the test sample is taken from the mother; and/or
b) taken from a pregnant control subject who later gave birth to a baby that is SGA, optionally wherein the control sample was taken from the control subject at substantially the same gestation or stage in pregnancy as the gestation or stage at which the test sample is taken from the mother.

8. The method of claim 1 wherein: (a) the test sample is a blood or plasma sample and/or (b) the test sample is taken from the mother at between around 12⁺⁰ weeks gestation and 21⁺⁶ weeks gestation, optionally between 12⁺⁰ and 14⁺⁶, or 15⁺⁰ and 17⁺⁶, or 18⁺⁰ and 21⁺⁶ weeks gestation.

9. (canceled)

10. The method of claim 1 wherein the level of the miRNA(s) is determined using: (a) digital molecular barcoding technology, optionally Nanostring technology, optionally the Nanostring nCounter miRNA profiling assay: or (b) RT-PCR, optionally wherein the method further comprises determining the amount of one or more suitable normalisation miRNAs present in the sample, optionally wherein the normalisation miRNAs are hsa-miR-30d-5p and/or hsa-let-7i-5p.

11. (canceled)

12. The method of claim 1 wherein the method comprises determining the amount of hsa-miR-374a-5p, hsa-let-7d-5p, hsa-miR-4454, and hsa-miR-7975 miRNAs using RT-PCR, optionally further comprises determining the level of hsa-miR-30d-5p and/or hsa-let-7i-5p miRNAs using RT-PCR.

13. The method according to claim 1 wherein the baby is predicted to be born at a birth-weight that is less than the 5^{th} centile for the particular gestational age at birth if the ratio of the amount of hsa-miR-374a-5p to hsa-miR-4454 is 1.5 or more, optionally at least 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9. 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0.

14. The method according to claim 1 wherein the baby is predicted to be born at a birth-weight that is less than the 5^{th} centile for the particular gestational age at birth if any one or more of the following criteria are met:
a) the amount of hsa-miR-374a-5p is increased relative to the amount of hsa-miR-374a-5p in a control sample, or is increased relative to a control value;
b) the amount of hsa-let-7d-5p is increased relative to the amount of hsa-let-7d-5p in a control sample, or is increased relative to a control value;
c) the amount of hsa-miR-191-5p is increased relative to the amount of hsa-miR-191-5p in a control sample, or is increased relative to a control value;
d) the amount of hsa-miR-4454 is decreased relative to the amount of hsa-miR-4454 in a control sample, or is decreased relative to a control value;
e) the amount of hsa-miR-7975 is decreased relative to the amount of hsa-miR-7975 in a control sample, or is decreased relative to a control value;
f) the amount of hsa-miR-107 is decreased relative to the amount of hsa-miR-107 in a control sample, or is decreased relative to a control value;
g) the amount of hsa-miR-30e-5p is decreased relative to the amount of hsa-miR-30e-5p in a control sample, or is decreased relative to a control value.

15. The method according to claim 1 wherein the baby is predicted to be born at a birth-weight that is less than the 5^{th} centile for the particular gestational age at birth when all of the following criteria are met:
a) the amount of hsa-miR-374a-5p is increased relative to the amount of hsa-miR-374a-5p in a control sample, or is increased relative to a control value;
b) the amount of hsa-let-7d-5p is increased relative to the amount of hsa-let-7d-5p in a control sample, or is increased relative to a control value;
c) the amount of hsa-miR-4454 is decreased relative to the amount of hsa-miR-4454 in a control sample, or is decreased relative to a control value; and
d) the amount of hsa-miR-7975 is decreased relative to the amount of hsa-miR-7975 in a control sample, or is decreased relative to a control value.

16. The method according to claim 1 wherein the baby is predicted to be born at a birth-weight that is less than the 5^{th} centile for the particular gestational age at birth when all of the following criteria are met:
a) the amount of hsa-miR-374a-5p is increased relative to the amount of hsa-miR-374a-5p in a control sample, or is increased relative to a control value;
b) the amount of hsa-let-7d-5p is increased relative to the amount of hsa-let-7d-5p in a control sample, or is increased relative to a control value;
c) the amount of hsa-miR-191-5p is increased relative to the amount of hsa-miR-191-5p in a control sample, or is increased relative to a control value;
d) the amount of hsa-miR-4454 is decreased relative to the amount of hsa-miR-4454 in a control sample, or is decreased relative to a control value;
e) the amount of hsa-miR-7975 is decreased relative to the amount of hsa-miR-7975 in a control sample, or is decreased relative to a control value;
f) the amount of hsa-miR-107 is decreased relative to the amount of hsa-miR-107 in a control sample, or is decreased relative to a control value; and
g) the amount of hsa-miR-30e-5p is decreased relative to the amount of hsa-miR-30e-5p in a control sample, or is decreased relative to a control value.

17. The method according to claim 1 wherein the method further comprises treating the mother and/or baby for SGA.

18. Nutritional supplements, anti-hypertension therapeutics, and/or aspirin for use in treating a baby and/or mother for determined or predicted birth at a birth-weight that is less than 5^{th} centile for the particular gestational age at birth, wherein the baby has been determined or predicted to be born at a birth-weight that is less than 5^{th} centile for the particular gestational age at birth using the method of the invention.

19. A kit comprising means to detect the amounts of any two or more of the following miRNAs: hsa-miR-374a-5p, hsa-let-7d-5p, hsa-miR-4454, hsa-miR-7975, hsa-miR-191, hsa-miR-107, or hsa-miR-30e-5p.

20. The kit of claim 19 wherein the kit comprises means to detect the amount of hsa-miR-374a-5p and hsa-miR-4454.

21. The kit of claim 19 wherein the kit comprises means to detect the amount of hsa-miR-374a-5p, hsa-let-7d-5p, hsa-miR-4454 and hsa-miR-7975.

22. The kit of claim 19 wherein the kit comprises means to detect the amount of all of hsa-miR-374a-5p, hsa-let-7d-5p, hsa-miR-4454, hsa-miR-7975, hsa-miR-191, hsa-miR-107, and hsa-miR-30e-5p.

23. The kit of claim 19 wherein the means to detect the amount of the miRNA are oligonucleotides, optionally wherein at least a part of the sequence of the oligonucleotide is complementary to at least part of the miRNA nucleic acid sequence; and optionally, wherein the means to detect the amount of the miRNAs are immobilised to a solid surface.

24. (canceled)
